(19) United States
(12) Patent Application Publication
(10) Pub. No.: US 2025/0334658 A1
(43) Pub. Date: Oct. 30, 2025

(54) FREQUENCY-AND-PHASE CORRECTION FOR MAGNETIC RESONANCE SPECTROSCOPY

(71) Applicant: The Trustees of Columbia University in the City of New York, New York, NY (US)

(72) Inventor: Jia GUO, Fort Lee, NJ (US)

(73) Assignee: The Trustees of Columbia University in the City of New York, New York, NY (US)

(21) Appl. No.: 19/072,214

(22) Filed: Mar. 6, 2025

Related U.S. Application Data
(63) Continuation of application No. PCT/US2023/073711, filed on Sep. 8, 2023.
(60) Provisional application No. 63/404,667, filed on Sep. 8, 2022.

(51) Int. Cl. G01R 33/46; G01R 33/485; G01R 33/56

(52) U.S. Cl. CPC G01R 33/4625; G01R 33/485; G01R 33/5608

(57) ABSTRACT

Implementations present convolutional neural network based spectral registration (CNN-SR) techniques that achieve efficient and accurate simultaneous frequency-and-phase correction (FPC) of magnetic resonance spectroscopy (MRS) data. Magnetic resonance spectroscopy research and clinical applications have provided invaluable information on the metabolic state of the brain. However, the data collection and analysis can be improved. For example, MRS data often undergoes correction after the data is collected, such as frequency correction and/or phase correction.

Implementations provide CNN-SR techniques to correct frequency and phase offset at the same time (e.g., simultaneously). The CNN-SR techniques leverages properties of a CNN that exploit spatial and temporal invariance in recognition of features, such as the overall shape of the signal and its peaks. Some embodiments perform model training in multiple phase and implement different training techniques (e.g., supervised training, unsupervised training, etc.) using different data sets.

This invention was made with government support under grant MH093398 awarded by the National Institutes of Health. The government has certain rights in the invention.

### FIELD

The embodiments of the present disclosure generally relate to frequency-and-phase correction for magnetic resonance spectroscopy.

### BACKGROUND

The metabolic profile of both human and animal brains may be non-invasively and quantitatively measured using magnetic resonance spectroscopy. The magnetic resonance spectroscopy research and clinical applications have provided invaluable information on the metabolic state of the brain. However, the data collection and analysis can be improved. For example, magnetic resonance spectroscopy data often undergoes correction after the data is collected, such as frequency correction and/or phase correction. Improvements to these analytical techniques can significantly improve the technological field and lead to an enhanced understanding of the metabolic state of human and animal brains.

### SUMMARY

The embodiments of the present disclosure generally relate to frequency-and-phase correction for magnetic resonance spectroscopy. Spectrum data generated using magnetic resonance spectroscopy is received, where the spectrum data relates to a subject's brain and a plurality of metabolite levels. Corrected spectrum data is generated by inputting the received spectrum data to a trained convolutional neural network, where the trained convolutional neural network simultaneously estimates frequency corrections and phase corrections for the input spectrum data. One or more metabolites are quantified using the corrected spectrum data.

Features and advantages of the embodiments are set forth in the description which follows, or will be apparent from the description, or may be learned by practice of the disclosure.

### BRIEF DESCRIPTION OF THE DRAWINGS

Further embodiments, details, advantages, and modifications will become apparent from the following detailed description of the preferred embodiments, which is to be taken in conjunction with the accompanying drawings.

FIG. 1 illustrates a system for performing frequency-and-phase correction of magnetic resonance spectroscopy data to quantify one or more metabolites according to example embodiment(s).

FIG. 2 illustrates a diagram of a computing system according to example embodiment(s).

FIGS. 3A and 3B are conceptual diagrams that illustrate example convolutional neural networks according to embodiment(s).

FIGS. 4 and 5 are visualizations of the performance of the deep learning models for frequency-and-phase correction according to a first example implementation.

FIGS. 6A, 6B, 7A, and 7B are a visual comparison between deep learning models for frequency-and-phase correction of On, OFF and Diff spectra according to a first example implementation.

FIG. 8 is a visual comparison of the variance of a choline interval in edited in vivo difference spectra among deep learning models with different levels of added offsets according to a first example implementation.

FIG. 9 is a visual comparison of model performance for an embodiment of the convolutional neural network based spectral registration model and a numerical method model-based spectral registration for in vivo datasets, in terms of the Off spectra, Diff spectra, performance scores, and variance of choline interval, according to a first example implementation.

FIG. 10 illustrates a table of mean absolute errors of deep learning model performance for frequency correction, phase correction, GABA residual, and Glx residual on the simulation dataset with different levels of noise according to a first example implementation.

FIG. 11 illustrates a table of model performance scores Q calculated between deep learning models under varying conditions according to a first example implementation.

FIG. 12 illustrates a table of choline residuals calculated using deep learning models under varying conditions according to a first example implementation.

FIGS. 13 and 14 illustrate visualizations of the performance of the deep learning models for frequency-and-phase correction according to a second example implementation.

FIGS. 15A, 15B, 16A, and 16B illustrate a visual comparison between deep learning models for frequency-and-phase correction of On, OFF and Diff spectra according to a second example implementation.

FIG. 17 illustrates a visual comparison of the variance of a choline interval in edited in vivo difference spectra among deep learning models with different levels of added offsets according to a second example implementation.

FIG. 18 illustrates a visual comparison of model performance for an embodiment of the convolutional neural network based spectral registration model and a numerical method model-based spectral registration for in vivo datasets, in terms of the Off spectra, Diff spectra, performance scores, and variance of choline interval, according to a second example implementation.

FIG. 19 illustrates a table of mean absolute errors of deep learning model performance for frequency correction, phase correction, GABA residual, and Glx residual on the simulation dataset with different levels of noise according to a second example implementation.

FIG. 20 illustrates a table of model performance scores Q calculated between deep learning models under varying conditions according to a second example implementation.

FIG. 21 illustrates a table of choline variances calculated using deep learning models under varying conditions according to a second example implementation.

FIG. 22 illustrates a flow diagram for performing frequency-and-phase correction of magnetic resonance spectroscopy data to quantify one or more metabolites according to example embodiment(s).

### DETAILED DESCRIPTION

Implementations present convolutional neural network based spectral registration (CNN-SR) techniques that achieve efficient and accurate simultaneous frequency-and-phase correction (FPC) of magnetic resonance spectroscopy (MRS) data. Spectral Registration is a technique to correct frequency and phase offsets. The algorithm is designed to align individual transients to a spectra template using a least square fitting method by maximizing the cross-correlation. This technique is often implemented in spectra editing software and applied on Magnetic Resonance Spectroscopy (MRS) data [See Cited References: 5, 3, 4]. MR Spectroscopy (MRS) is an analytical tool used to quantify metabolic chemical changes in human and animal brains, which can provide crucial information on brain health. However, because MR is sensitive to scanner variabilities, such as frequency drift and subject motion during scanning, frequency and phase shifts may arise that impact the data analysis. Frequency-and-phase correction (FPC) can improve the accuracy of spectral registration and metabolite quantification.

For instance, the metabolite Gamma-aminobutyric acid (GABA) is the primary inhibitory neurotransmitter in the human brain, but its concentration can be challenging to quantify due to the overlapping metabolite Creatine (Cr), which is present in much greater concentrations. Among a range of techniques to assess GABA in vivo, Meshcher-Garwood point-resolved spectroscopy (MEGA-PRESS) is a widely used MRS technique [See Cited References: 6, 8, 2]. MEGA PRESS is a J-difference editing (JDE) pulse sequence that separates overlapping metabolites from each other. However, a limitation in JDE pulse sequences is the reliance on the subtraction of spectral edited “On Spectra” and non-edited “Off Spectra” to reveal the edited resonance in the “Diff Spectra”.

As a result of the overlapping resonances being an order of magnitude larger in intensity than the GABA resonance, changes in scanner frequency and spectral phase can cause incomplete subtraction in the edited spectrum. Small changes in scanner frequency can possibly arise from gradient-induced heating of passive shim elements and long time-constant eddy currents while small changes in spectral phase shift can possibly arise from respiratory-induced magnetic field drifts [See Cited References: 2,7,11]. A standard approach in GABA editing is to apply frequency and phase drift correction of individual frequency domain transients by fitting the Cr signal at 3 ppm [See Cited References: 6,9]. A limitation of the Cr fitting-based correction method, however, is that it relies strongly on sufficient signal-to-noise ratio (SNR) of the Cr signal in the spectrum. To over-come this limitation, some SR approaches were proposed that can accurately align single transients in the time domain or frequency domain [See Cited References: 3-5]. However, the correction accuracy largely depends on the overall spectral SNR where low SNR (i.e., 2.5) will deteriorate the performance as the signal will be dominated by noise [See Cited References: 7]. Furthermore, medical applications for metabolite quantification of this kind would greatly benefit from more robust, fast, and high registration accuracy technique(s).

Deep learning has become a popular technique used to address complex computational challenges, and deep learning has, at times, been an effective and successful image processing tool adopted in medical image registration [See Cited References: 12, 13]. The learning-based registration method presented by embodiments of this disclosure optimizes a global function for a dataset during training, thereby limiting time-consumption and computationally expensive per-image optimization during inference.

A multilayer perceptron (MLP) model [See Cited References: 14] and a convolutional neural network (CNN) model [See Cited References: 10] have been recently applied to single-transient sequential FPC for edited MRS. Both of these models (MLP-FPC and CNN-FPC) demonstrate the potential of applying deep learning in MRS data processing by pre-training models with simulated datasets with wide ranges of frequency and phase offsets. Although both these models yield well-predicted results, the utility in spectral registration is limited due to the models' requirement to be separately trained for frequency and phase offset prediction, and separately used to perform FPC. A limitation in this training is that the subtraction errors caused by phase and frequency errors appear similar but require different corrections. If the error is misdiagnosed, an improper correction will be applied, which may degrade the quality of spectral subtraction. Therefore, a more efficient network that can mimic the nature of performing simultaneous FPC similarly to the spectral editing techniques could be considered to more accurately perform FPC of the given data. Implementations provide a CNN spectral registration technique (CNN-SR) to correct frequency and phase offset at the same time (e.g., simultaneously) while comprising the CNN properties of exploiting spatial and temporal invariance in recognition of features such as the overall shape of the signal and its peaks.

Implementations demonstrate the utility of CNNs for spectral registration, for example using single voxel MEGA-PRESS MRS data. An embodiment of the CNN spectral registration (CNN-SR) technique performs simultaneous FPC. This embodiment of the CNN-SR approach was tested on a published simulated dataset and an in vivo dataset against benchmark neural network approaches using MLP and CNN [See Cited References: 14, 10]. The testing demonstrated that this embodiment achieved superior performance when compared to MLP-FPC and CNN-FPC.

An embodiment of the CNN-SR technique was tested using MRS data with additional noise and line broadening of SNR 2.5 and 0-20 ms, respectively. This testing further demonstrated the utility of the CNN-SR technique in the presence of a more distorted spectra. An embodiment of the CNN-SR technique was also tested using in vivo MRS data with different magnitudes of additional offsets (e.g., none, small, moderate, large) to further demonstrate the utility of the CNN-SR technique to accurately predict the spectral frequency and phase offsets.

Further, an embodiment of the CNN-SR technique incorporates an unsupervised learning spectral registration approach. For example, the unsupervised learning spectral registration approach was applied on the in vivo data. The CNN-SR technique that incorporates the unsupervised learning spectral registration is referred to as CNN-SR+ in this disclosure. With respect to the testing incorporated by embodiments in this disclosure, the CNN-SR+ technique(s) performed better than SR techniques, and the CNN-SR technique(s) had similar performances with the published numerical method model-based SR (mSR) [See Cited References: 3].

Reference will now be made in detail to the embodiments of the present disclosure, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, and circuits have not been described in detail so as not to unnecessarily obscure aspects of the embodiments. Wherever possible, like reference numbers will be used for like elements.

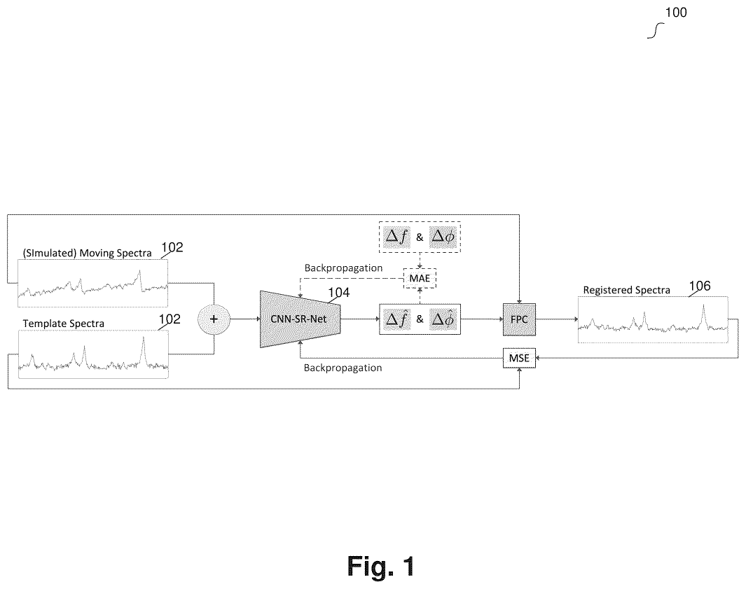

FIG. 1 illustrates a system for performing frequency-and-phase correction of magnetic resonance spectroscopy data to quantify one or more metabolites according to example embodiment(s). System 100 illustrates data sets 102, network 104, and registered spectra 106. Components of system 100 can accomplish training of network 104, for example using a loss function and gradient propagation via backpropagation. Network 104 can be a convolutional neural network, such as the CNN-SR and CNN-SR+ embodiments disclosed herein. Data sets 102 can include any suitable data sets for training (e.g., supervised training, unsupervised training, etc.) of network 104. Network 104 can be trained to simultaneously estimate frequency-and-phase correction to generate registered spectra 106.

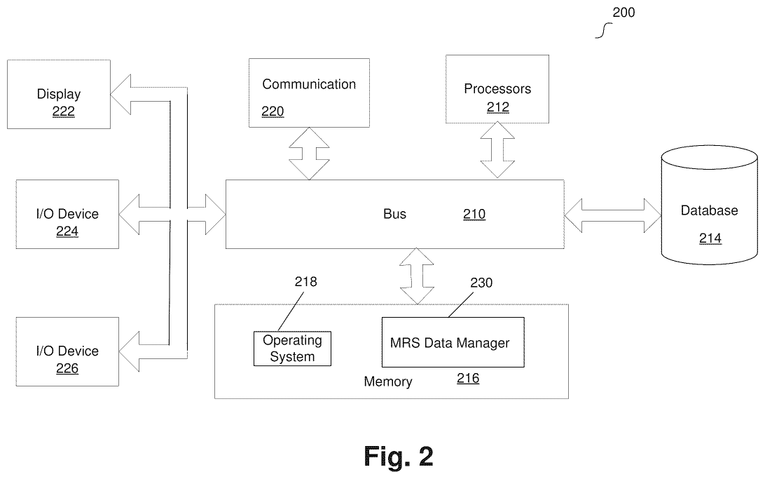

FIG. 2 is a diagram of a computing system 200 in accordance with embodiments. As shown in FIG. 2, system 200 may include a bus 210, as well as other elements, configured to communicate information among processor 212, data 214, memory 216, and/or other components of system 200. Processor 212 may include one or more general or specific purpose processors configured to execute commands, perform computation, and/or control functions of system 200. Processor 212 may include a single integrated circuit, such as a micro-processing device, or may include multiple integrated circuit devices and/or circuit boards working in combination. Processor 212 may execute software, such as operating system 218, MRS data manager 230, and/or other applications stored at memory 216.c

Communication component 220 may enable connectivity between the components of system 200 and other devices, such as by processing (e.g., encoding) data to be sent from one or more components of system 200 to another device over a network (not shown) and processing (e.g., decoding) data received from another system over the network for one or more components of system 200. For example, communication component 220 may include a network interface card that is configured to provide wireless network communications. Any suitable wireless communication protocols or techniques may be implemented by communication component 220, such as Wi-Fi, Bluetooth®, Zigbee, radio, infrared, and/or cellular communication technologies and protocols. In some embodiments, communication component 220 may provide wired network connections, techniques, and protocols, such as an Ethernet.

System 200 includes memory 216, which can store information and instructions for processor 212. Embodiments of memory 216 contain components for retrieving, reading, writing, modifying, and storing data. Memory 216 may store software that performs functions when executed by processor 212. For example, operating system 218 (and processor 212) can provide operating system functionality for system 200. MRS data manager 230 (and processor 212) can correct frequency and phase of MRS data for metabolite quantification. Embodiments of MRS data manager 230 may be implemented as an in-memory configuration. Software modules of memory 216 can include operating system 218, MRS data manager 230, as well as other applications modules (not depicted).

Memory 216 includes non-transitory computer-readable media accessible by the components of system 200. For example, memory 216 may include any combination of random access memory (“RAM”), dynamic RAM (“DRAM”), static RAM (“SRAM”), read only memory (“ROM”), flash memory, cache memory, and/or any other types of non-transitory computer-readable medium. A database 214 is communicatively connected to other components of system 200 (such as via bus 212) to provide storage for the components of system 200. Embodiments of database 214 can store data in an integrated collection of logically-related records or files.

Database 214 can be a data warehouse, a distributed database, a cloud database, a secure database, an analytical database, a production database, a non-production database, an end-user database, a remote database, an in-memory database, a real-time database, a relational database, an object-oriented database, a hierarchical database, a multi-dimensional database, a Hadoop Distributed File System (“HFDS”), a NoSQL database, or any other database known in the art. Components of system 200 are further coupled (e.g., via bus 210) to: display 222 such that processor 212 can display information, data, and any other suitable display to a user, I/O device 224, such as a keyboard, and I/O device 226 such as a computer mouse or any other suitable I/O device.

In some embodiments, system 200 can be an element of a system architecture, distributed system, or other suitable system. For example, system 200 can include one or more additional functional modules, which may include the various modules of data analytics processing tools, machine learning libraries, MRS analytics toolkit(s), Matlab® modules, MEGA-PRESS software modules, or any other suitable modules.

Embodiments of system 200 can remotely provide the relevant functionality for a separate device. In some embodiments, one or more components of system 200 may not be implemented. For example, system 200 may be a tablet, smartphone, or other wireless device that includes a display, one or more processors, and memory, but that does not include one or more other components of system 200 shown in FIG. 2. In some embodiments, implementations of system 200 can include additional components not shown in FIG. 2. While FIG. 2 depicts system 200 as a single system, the functionality of system 200 may be implemented at different locations, as a distributed system, within a cloud infrastructure, or in any other suitable manner. In some embodiments, memory 216, processor 212, and/or database 214 are be distributed (across multiple devices or computers that represent system 200). In one embodiment, system 200 may be part of a computing device (e.g., smartphone, tablet, computer, and the like).

This disclosure describes two example implementations: a) a first example implementation of a CNN-SR embodiment and a CNN-SR+ embodiment; and b) a second example implementation of a CNN-SR+ embodiment. In both example implementations, the performance of embodiments is tested against other model performance.

### First Example Implementation of CNN-SR and CNN-SR+

In some implementations, the MRS data that undergoes frequency-and-phase correction can be single-voxel MEGA-PRESS MRS data. Implementations include a neural network that is trained and validated. In one embodiment, an example neural network was trained and validated using a simulated data set and an in vivo MEGA-PRESS MRS dataset with a wide-range of artificial frequency (0-20 Hz) and phase (0-90°) offsets applied. The embodiment of the CNN-SR approach was subsequently tested and compared to sequential FPC deep learning approaches, and the embodiment demonstrated more effective and accurate performance.

Further, random Gaussian signal-to-noise ratio (SNR 20 and SNR 2.5) and line broadening (0-20 ms) was introduced to the original simulated dataset to investigate the experimental implementation as compared to the other deep learning models. The testing showed that the experimental implementation of the CNN-SR techniques was a more accurate quantification tool and resulted in a lower SNR when compared with the other deep learning methods, due to having smaller mean absolute errors in both frequency and phase offset predictions.

For Off spectra, the experimental implementation of the CNN-SR techniques was capable of correcting frequency offsets with 0.014±0.010 Hz and phase offsets with 0.104±0.076° absolute errors on average for unseen simulated data with SNR 20 and correcting frequency offsets with 0.678±0.883 Hz and phase offsets with 2.367±2.616° absolute errors on average at very low SNR (2.5) and line broadening (0-20 ms) introduced.

A further refined experimental implementation tested the simulated dataset with additional SNR and line broadening using a pre-trained CNN-SR that was further optimized by using unsupervised learning to minimize a difference between individual spectra and a common template. The performance of the refined experimental implementation on Off spectra was improved to 0.058±0.050 Hz for correcting frequency offsets and to 0.416±0.317° for correcting phase offsets.

Some embodiments were also used to process the published Big GABA in vivo dataset, and the CNN-SR+ embodiment achieved the best performance. Moreover, additional frequency and phase offsets (i.e., small, moderate, large) were applied to the in vivo dataset, and the CNN-SR+ embodiment also demonstrated better performance for FPC when compared to the other deep learning models. These experimental implementations demonstrate the utility of using CNN-SR+ for spectral registration. In addition, some implementations further demonstrate the application of unsupervised learning to improve model performance in certain scenarios.

### First Example Implementation of CNN-SR and CNN-SR+: Data Sets

### Simulated Data Sets

One challenge deep learning techniques often experience is determining the inputs and ground truth for model training, for example to achieve a specific performance goal. Since the ground truth of frequency and phase offsets for the in vivo dataset is not available, embodiments simulated the MEGA-PRESS training, validation, and test transients using an FID-A toolbox (version 1.2) in Matlab, with the same parameters as described in the previous work [See Cited References: 14, 10]. The training set was allocated 32,000 OFF+ON spectra, and 4,000 for both validation set and test set. Other suitable set breakdowns can be implemented. Embodiments were tested using datasets with added random Gaussian noise at SNR 20, and testing in some embodiments involved lower SNR 2.5 and Line Broadening (0-20 ms). The SNR values were computed by the ratio of the Cr peak signal relative to the noise standard deviation.

### In Vivo Data Sets

In vivo data involved in some embodiments was retrieved from the publicly available Big GABA repository [See Cited References: 15]. In addition, 101 MEGA-edited datasets from nine sites with Philips scanners were collected for embodiments, where each dataset contained 320 transients OFF+ON. Some embodiments were also evaluated on this in vivo dataset with additional offsets (e.g., small, medium, large).

### First Example Implementation of CNN-SR and CNN-SR+: Network Architecture

Embodiments incorporate both supervised and unsupervised learning in the proposed CNN-SR techniques on the simulation dataset. Some embodiments further fine tune this training using unsupervised learning on the in vivo dataset. During supervised training, both supervised loss and unsupervised loss were implemented to optimize the network parameters, as illustrated in FIG. 1. Based on the CNN-SR model, CNN-SR+ model used unsupervised loss for training related to the in vivo dataset.

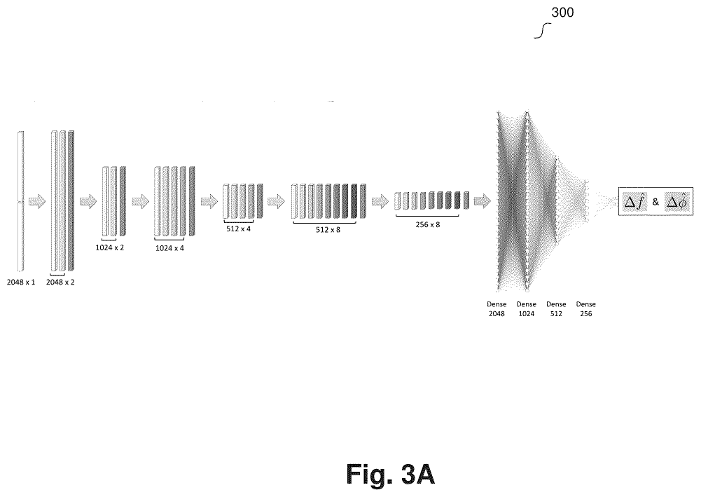

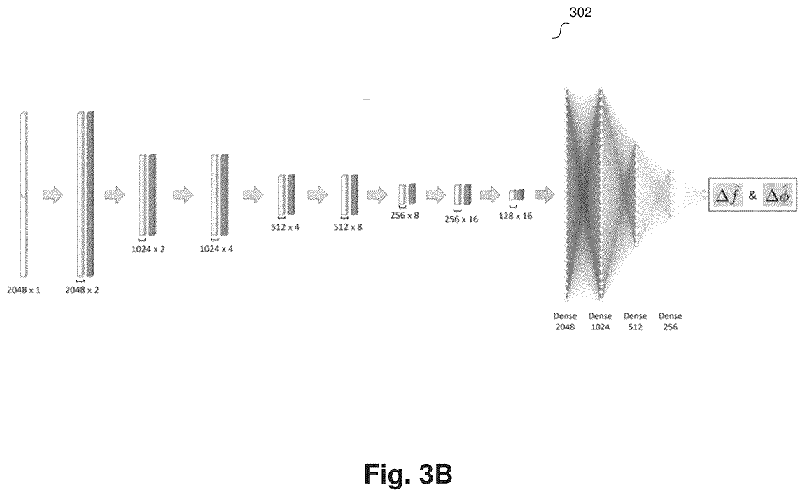

FIG. 3A illustrates an example neural network according to some embodiments. Network 300 illustrates a sequential network that takes moving spectra and template spectra as inputs and predicts frequency and phase offsets at the same time (e.g., simultaneously). In some implementations, both moving spectra and template spectra are processed to have length of 1024 and are concatenated to form a single 2048 input array. Other suitable sizes, orientations, and dimensions can be implemented.

Network 300 starts with successive layers (e.g., three or four), each comprising a one dimensional convolutional layer followed by a one-dimensional max-pooling layer. The convolutional layer comprises of 4 kernels with a size of 3, and the max-pooling layer has a pool size of 2 with a stride of 2. Other suitable network architectures, parameters, or orientations can be implemented.

Network 300 also includes fully-connected layers (FC) with 1024, 512 and 256 nodes, and a final fully-connected linear output layer of two nodes. In the illustrated network 400, each hidden layer was followed by a rectified linear unit (ReLU) activation function to introduce non-linearity. An Adam optimizer [See Cited References: 16] was used to train the neural network with a 0.0001 learning rate in some embodiments. The output from network 400 is predicted offsets of frequency and phase. In some embodiments, model(s) were trained for 300 epochs with a batch size of 32, and the mean absolute error was used as the loss function. Any other suitable parameters can be implemented.

### Network Testing

On the scale of −20 to 20 Hz and −90° to 90°, uniformly distributed artificial offsets were added to simulated spectra to generate input moving spectra in some embodiments, comprising frequency drift and a phase drift. Different level(s) of Gaussian distributed noise and Line Broadening were added to the moving spectra prior inputting into the network in some embodiments. First, some embodiments applied a Fast Fourier transform to the uncorrected moving spectra and normalized them to the maximum signal in the spectrum. In an example technique, the peripheral 1024 samples were cropped off, and the central 1024 samples were selected and absolute value was taken to feed the network. The same normalization and cropping can be applied to moving spectra using FPC to generate the registered spectra. Any other suitable selection technique to feed the network and/or technique to generate registered spectra can be implemented.

### Example Implementation of CNN-SR and CNN-SR+: Evaluation and Comparison Using In Vivo Dataset

In some embodiments, the MEGA-edited datasets were used as the test set of the CNN SR network(s). In a first comparison of the performance of the CNN-SR network(s), a published model-based SR (mSR) [See Cited References: 3], a non-deep learning approach, was used to perform FPC in the time domain. mSR uses a noise-free model as the template instead of the median transient of the dataset. Noise-free ON and OFF FID models were created in Osprey (version 1.0.0), an open-source MatLab toolbox, following peer-reviewed preprocessing recommendations [See Cited References: 15]. Embodiments of the CNN-SR model(s) were also compared to a benchmark neural network comprising 3 FC layers (1024, 512, 1 node(s)) [See Cited References: 14] and a CNN comprising two convolutional blocks (e.g., convolutional layer with 4 kernels of size 3+Max pooling layer with downsampling size 2 stride 2) and 3 FC layers (e.g., 1024, 512, 1 node(s)) [See Cited References: 10]. In both of these networks, each hidden FC layer was followed by a ReLU activation function, and a linear activation function followed the output layer.

To examine the networks under test in different environments, additional series of artificial offsets were added to the in vivo data. Examples included three different kinds of additionally added offsets: 1. 0≤|Δf|≤5 Hz and 0°≤|Δϕ|≤20°; 2. 5≤|Δf|10 Hz and 20°≤|Δϕ|≤45°; 3. 163 10≤|Δf|≤20 Hz and 45°≤|Δϕ|≤90°. The additional offsets were sampled from a uniform distribution and added as random pairs of frequency and phase to each transient.

### First Example Implementation of CNN-SR and CNN-SR+: Hardware and Software

Testing on embodiments was conducted with an Intel® Xeon® CPU E5-2650 v4 @2.20 GHz processor and an NVIDIA GeForce RTX 2080 Ti GPU with a memory of 11 GB.

### First Example Implementation of CNN-SR and CNN-SR+: Performance Measurements

In the simulated dataset, the artificial offsets were set as the ground truth, and the mean absolute error between the ground truth and predicted value was used as the criteria to measure the networks' performance that was under test. The difference value between the true spectra and the corrected spectra using mSR, MLP-FPC, CNN—FPC and CNN-SR was calculated and plotted. A Q score [See Cited References: 14] was used to determine the performance strengths of each different technique, and it is defined as Q=1−σ₁²/(σ₁²+σ₂²), where σ² is the variance of the choline subtracted artifact in the average difference spectrum. If the Q score is greater than 0.5, it indicates that the first method performs better than the second method and vice versa.

### First Example Implementation of CNN-SR and CNN-SR+: Results

### Visual Comparison of Model Performance

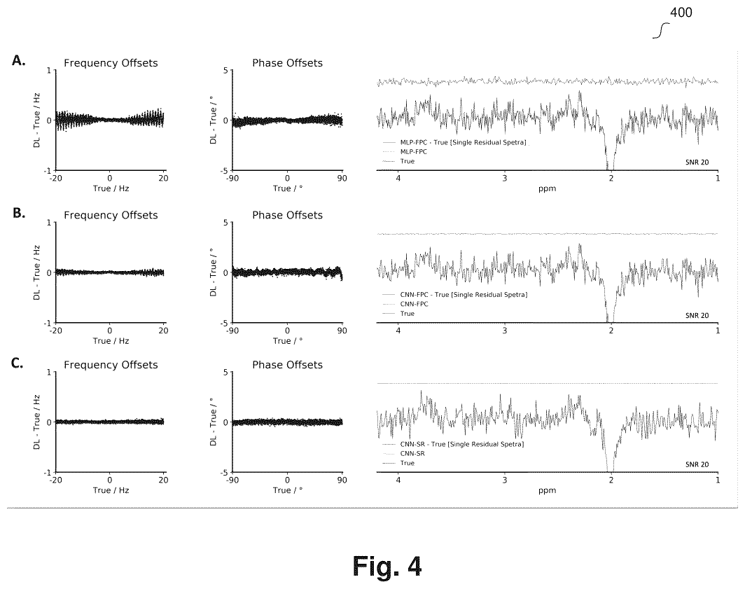

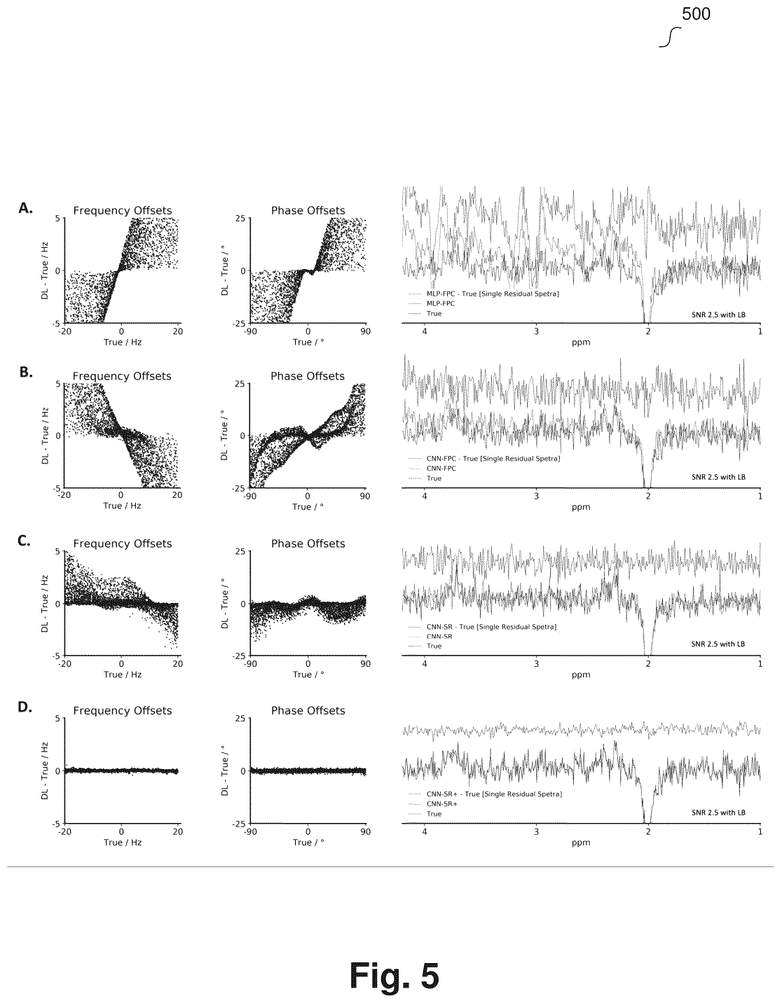

FIGS. 4 and 5 are visualizations of the performance of the deep learning models for frequency-and-phase correction. Diagram 400 illustrates a visualization of the performance of the deep learning models (MLP-FPC, CNN-FPC, CNN-SR) for frequency-and-phase correction using the published simulated dataset with added noise at the SNR of 20. For the MLP-FPC, the CNN-FPC and the CNN-SR model, the scatter plots on the left of diagram 400 show the correction errors between the ground truths and model predictions at different frequency and phase offsets. The spectra on the right of diagram 400 demonstrate the spectrum predicted by each deep learning model, the true MEGA-PRESS difference spectra, and the subtraction between them. Among all 3 models, the MLP-FPC exhibits larger correction errors for frequency and phase offset followed by the CNN-FPC, with both being outperformed by the CNN-SR. Diagram 400 illustrates: (A) Output of the MLP-FPC model on the simulated dataset; (B) Output of the CNN-FPC model on the simulated dataset; (C) Output of the CNN-SR model on the simulated dataset.

Diagram 500 illustrates a visualization of the performance of the deep learning models (MLP-FPC, CNN-FPC, CNN-SR, CNN-SR+) for frequency-and-phase correction using the published simulated dataset with line broadening and added noise at the SNR of 2.5. For the MLP-FPC, the CNN-FPC, the CNN-SR and the CNN-SR+ models, the scatter plots on the left of diagram 500 show the correction errors between the ground truths and model predictions at different frequency and phase offsets. The spectra on the right of diagram 500 demonstrate the spectrum predicted by each deep learning model, the true MEGA-PRESS difference spectra, and the subtraction between them. Among all 4 models, the MLP-FPC exhibits larger correction errors for frequency and phase offset followed by the CNN-FPC and the CNN-SR, with all being outperformed by the CNN-SR+. Diagram 500 illustrates: (A) Output of the MLP-FPC model on the simulated dataset; (B) Output of the CNN-FPC model on the simulated dataset; (C) Output of the CNN-SR model on the simulated dataset; (D) Output of the CNN-SR+ model on the simulated dataset.

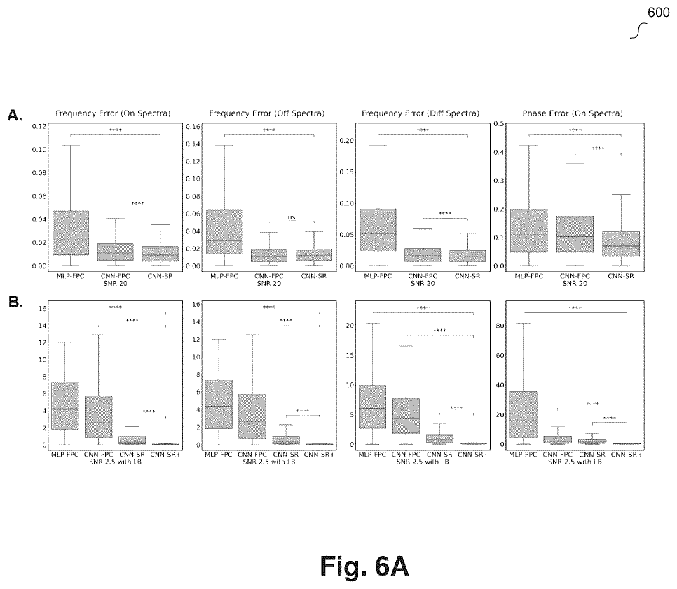

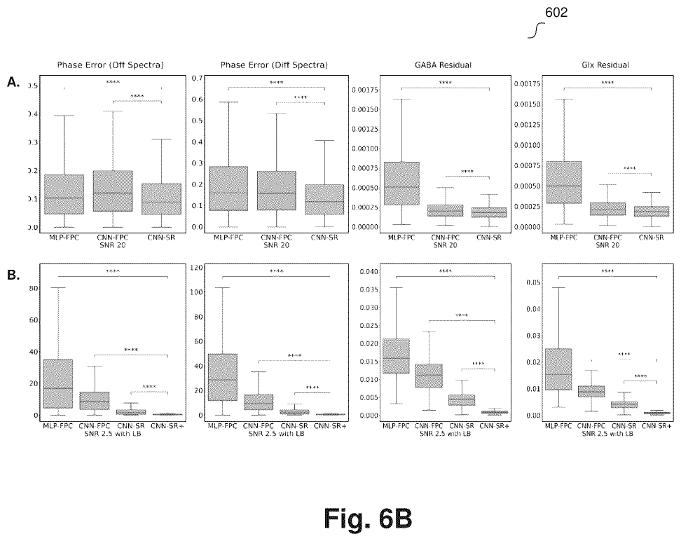

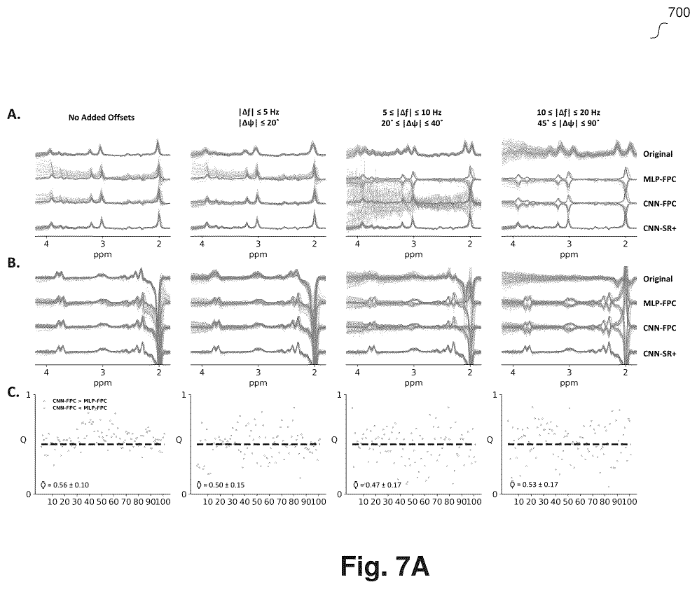

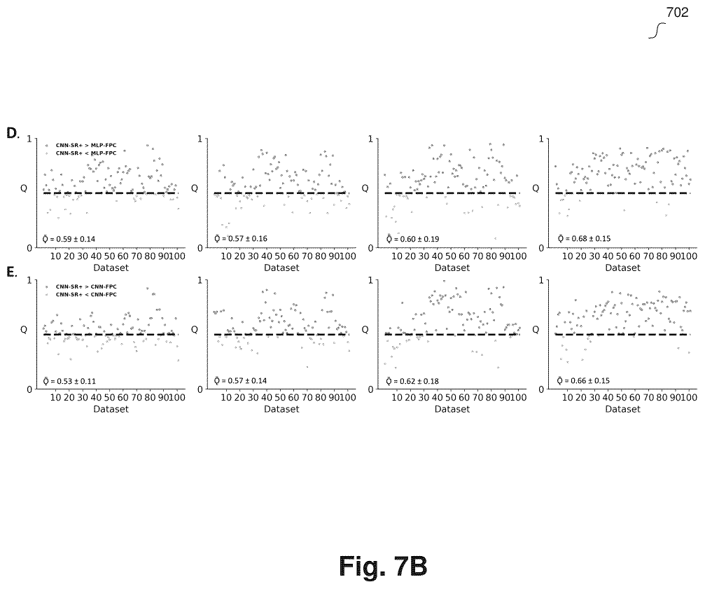

FIGS. 6A, 6B, 7A and 7B are a visual comparison between deep learning models for frequency-and-phase correction of On, OFF and Diff spectra. Diagrams 600 and 602 illustrate a comparison between the MLP-FPC model, the CNN-FPC model and the CNN-SR embodiment for frequency-and-phase correction of the On, OFF and Diff spectra at the SNR of 20 and the SNR of 2.5 with line broadening. From left to right, diagrams 600 and 602 show: the frequency estimation error of the On spectra, the frequency estimation error of the Off spectra, the frequency estimation error of the Diff spectra, the phase estimation error of the On spectra, the phase estimation error of the Off spectra, the phase estimation error of the Diff spectra, the GABA residual spectra mean absolute error, and the Glx residual spectra mean absolute error. Diagrams 600 and 602 include: (A) Box plots showing the frequency estimation error (in Hz), the phase estimation error (in degrees) and the GABA and the Gix residual spectra mean absolute error of the MLP-FPC model, the CNN-FPC model and the CNN-SR model at the SNR of 20; (B) Box plots showing the frequency estimation error (in Hz), the phase estimation error (in degrees) and the GABA and the Glx residual spectra mean absolute error of the MLP-FPC model, the CNN-FPC model and the CNN-SR model at the SNR 2.5 with line broadening. With respect to the symbols “****” in diagrams 600 and 602: The two-tailed p-value is less than 0.0001.

Diagrams 700 and 702 illustrate the in vivo Off and Diff spectra results of models with different level of added offsets and performance scores comparing the CNN-FPC model to the MLP-FPC model, the CNN-SR+ embodiment to the MLP-FPC model, and the CNN-SR+ embodiment to the CNN-FPC model for the 101 in vivo datasets. Diagrams 700 and 702 include: (A) The original Off spectra and the results of 3 models after applying corrections to the in vivo data without further manipulation and with additional frequency and phase offsets applied to the same 101 datasets: small offsets (0-5 Hz; 0-20°), moderate offsets (5-10 Hz; 20-40°), and large offsets (10-20 Hz; 45-90°); (B) The original Diff spectra and the results of 3 models after applying corrections to the in vivo data without further manipulation and with additional frequency and phase offsets applied to the same 101 datasets: small offsets (0-5 Hz; 0-20°), moderate offsets (5-10 Hz; 20-40°), and large offsets (10-20 Hz; 45-90°); (C) Comparative performance Q scores for the CNN-FPC model and the MLP-FPC model for each dataset. A score above 0.5 indicated that the CNN-FPC model performed better than the MLP-FPC model in terms of alignment, whereas a score below 0.5 indicated the opposite. (D) Comparative performance Q scores for the CNN-SR+ embodiment and the MLP-FPC model for each dataset. (E) Comparative performance Q scores for the CNN-SR+ embodiment and the CNN-FPC model for each dataset.

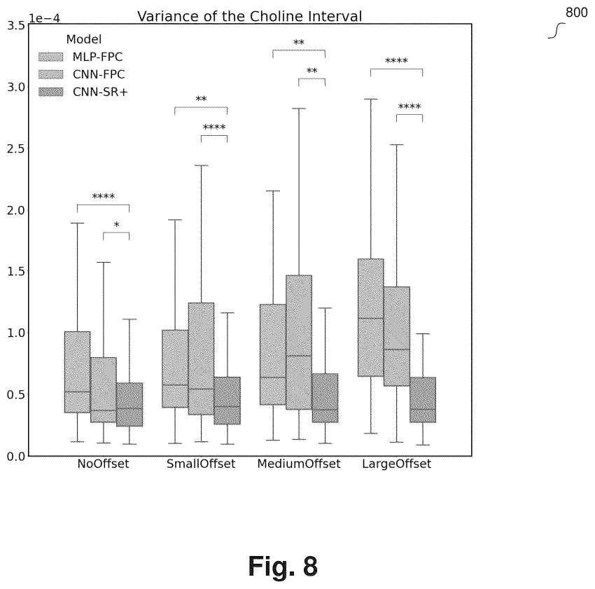

FIG. 8 is a visual comparison of the variance of a choline interval in edited in vivo difference spectra among deep learning models with different levels of added offsets. Diagram 800 illustrates a comparison of the variance of the choline interval in the edited in vivo difference spectra among the MLP-FPC model, CNN-FPC mode, and CNN-SR+ embodiment with different levels of added offsets. From left to right, diagram 800 includes: box plots of choline interval variances with no offset, small offsets, medium offsets and large offsets. The CNN-SR+ embodiments has relatively stable performance and its generated variance of the choline interval is significantly lower than both the MLP-FPC model and the CNN-FPC model at all offset levels. With no offset or large offset, the CNN-FPC model has lower choline interval variance than the MLP-FPC model; but with small or medium offsets, the MLP-FPC model has lower choline interval variance than the CNN-FPC model. With respect to the following symbols in diagram 800: “****”—The two-tailed p-value is less than 0.0001; “**”—The two-tailed p-value is between 0.001 and 0.01; “*”—The two-tailed p-value is between 0.01 and 0.05.

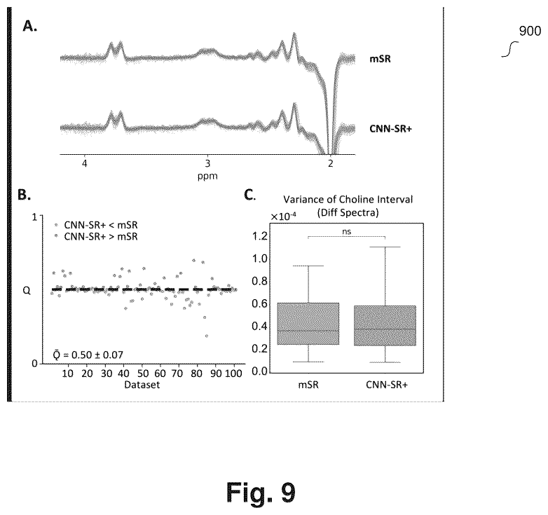

FIG. 9 is a visual comparison of model performance comparison an embodiment of the convolutional neural network based spectral registration model and a numerical method model-based spectral registration for in vivo datasets, in terms of the Off spectra, Diff spectra, performance scores, and variance of choline interval. Diagram 900 illustrates model performance comparison between the CNN-SR+ embodiment and the mSR model for in vivo datasets, in terms of the Off spectra, Diff spectra, performance scores and the variance of choline interval. Diagram 900 includes: (A) The Diff spectra results of the CNN-SR+ embodiment and the mSR model; (B) Comparative performance scores Q for the CNN-SR+ embodiment and the mSR model for each dataset. A score above 0.5 indicated that the CNN-SR+ embodiment performed better than the mSR model in terms of alignment, whereas a score below 0.5 indicated the opposite. (C) Box plots of the variance of the choline interval of the CNN-SR+ embodiment and the mSR model, showing no significant difference.

### Model-Performance Evaluation and Spectra Analysis for the Simulated Datasets

The results of the MLP-based approach and CNN-based approaches on the simulated test dataset with lower SNR of 20 and SNR of 2.5 with line broadening are illustrated in FIGS. 4 and 5. In each subfigure, the frequency offset errors are plotted against their corresponding correct values, the phase offset errors are plotted against their corresponding correct values, the model-corrected spectrum and the difference spectrum corrected by the true offsets are plotted together, and the residues between the difference spectra are shown. The comparison of the errors for FPC of the MLP-based approach and the CNN-based approach of the On, Off and Diff spectra of the simulated test set for varying SNRs is illustrated in FIGS. 6A and 6B.

For the test set with SNR 20, the CNN-based approaches showed significantly lower frequency estimation errors than the MLP-based approach, and the CNN-SR embodiment showed the lowest phase estimation errors for the On, Off and Diff spectra (as illustrated in FIGS. 6A-6B—Diagram A). Taking the Diff spectra as an example, the mean frequency offset error was 0.064±0.052 Hz for the MLP-FPC model, 0.020±0.016 Hz for the CNN-FPC model, and 0.017±0.013 Hz for the CNN-SR model. The mean phase offset error was 0.197±0.159° for the MLP-FPC model, 0.186±0.142° for the CNN-FPC model, and 0.137±0.100° Hz for the CNN-SR embodiment.

With a lower SNR at 2.5 with random 0-20 ms line broadening (As illustrated in FIGS. 6A-6B—Diagram B), the CNN-SR+ embodiment showed significantly lower frequency and phase estimation errors than the other models for the On, Off and Diff spectra. For example, the mean frequency offset and phase estimation errors for the Diff spectra was 6.658±4.734 Hz, 33.760±26.863° for the MLP-FPC model, 5.264±4.170 Hz, 11.824±9.630° for the CNN-FPC model, 1.067±1.061, 2.987±2.662° Hz for the CNN-SR model and 0.080±0.065, 0.554±0.426° for the CNN-SR+ embodiment.

These results in FIGS. 4 and 5 show that compared to the MLP-based approaches, the CNN-based models had smaller errors within the frequency and phase ranges tested. At the SNR of 20, the CNN-SR embodiment performed better than the MLP-FPC model and the CNN-FPC model. When the SNR decreased to 2.5 and line broadening is applied, the CNN-SR+ embodiment performed better than the MLP-FPC model, CNN-FPC model, and CNN-SR embodiment that had less stable predictions and larger errors.

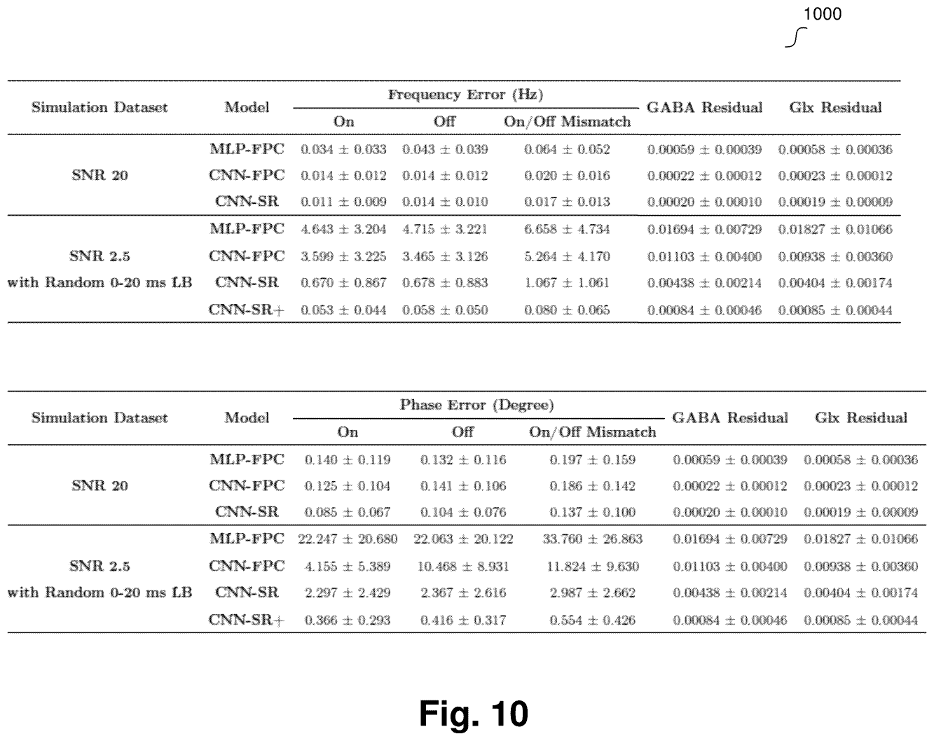

Additionally, by extracting the spectra interval corresponding to GABA (i.e., 2.8-3.2 ppm) and Glx (i.e., 3.55-3.95 ppm) from the derived mean difference spectra (As illustrated in FIGS. 6A-6B), these residual spectra errors were found to be lower with the CNN-SR embodiment (at the SNR of 20) and CNN-SR+ embodiment (at the SNR of 2.5 with line broadening) than the MLP-FPC and CNN-FPC models. Consequently, the residual spectra errors using CNN-based models for the full spectra were significantly lower than those of the MLP-based model for the On, OFF and Diff spectra at a lower SNR, indicating CNN based models' higher performance and robustness in the presence of noise with respect to the MLP-FPC model. Among CNN-based models, the CNN-SR+ embodiment performed the best in terms of frequency and phase estimation errors and noise tolerance, followed by the CNN-SR embodiment (numerical results are shown in table 1000 of FIG. 10).

### Model-Performance Evaluation and Spectra Analysis for the In Vivo Big GABA Datasets

FIG. 7A Diagrams A and B illustrate the Off and Diff spectra resulting from the 131 in vivo Big GABA Philips datasets without (column 1) or with (columns 2-4) additional artificial offsets for no correction, MLP-FPC model correction, CNN-FPC model correction, and CNN-SR+ embodiment correction. The additional frequency and phase offsets applied to the same 101 datasets are small offsets (e.g., 0-5 Hz; 0-20°), moderate offsets (e.g., 5-10 Hz; 20-45°), and large offsets (e.g., 10-20 Hz; 45-90°). FIG. 7A Diagram C, FIG. 7B Diagram D, and FIG. 7B Diagram 5E demonstrate performance scores comparing the CNN-FPC model to the MLP-FPC model, the CNN-SR+ embodiment to the MLP-FPC model and the CNN-SR+ embodiment to the CNN-FPC model for the 101 in vivo datasets.

When small offsets were added, the MLP-FPC and CNN-FPC models performed similarly (mean performance score 0.50±0.15, as illustrated in FIG. 7A Diagram C, column 2), and both were outperformed by the CNN-SR+ embodiment. The mean performance score of the CNN-SR+ embodiment against the MLP-FPC model was 0.57±0.16 (FIG. 7B Diagram D, column 2) and was 0.57±0.14 for the CNN-SR+ embodiment against the CNN-FPC model (as illustrated in FIG. 7B Diagram E, column 2).

As for moderate offsets, the performance of the MLP-FPC model and the CNN-FPC model was comparable, but the CNN-SR+ embodiment still performed better. The mean performance score of the CNN-FPC model against the MLP-FPC model was 0.47±0.17 (FIG. 7A Diagram C, column 3), while it was 0.60±0.19 for the CNN-SR+ embodiment against the MLP-FPC model (As illustrated in FIG. 7B Diagram D, column 3), and 0.62±0.18 for the CNN-SR+ embodiment against the CNN-FPC model (As illustrated in FIG. 7B Diagram E, column 3).

When large offsets were added, the performance of the CNN-FPC model was slightly better than the MLP-FPC model. The CNN-SR+ embodiment still outperformed the MLP FPC and CNN-FPC models. The mean performance score of the CNN-FPC model against the MLP-FPC model was 0.53±0.17 (As illustrated in FIG. 7A Diagram C, column 4), while it was 0.68±0.15 for the CNN-SR+ embodiment against the MLP-FPC model (As illustrated in FIG. 7B Diagram D, column 4), and 0.66±0.15 for the CNN-SR+ embodiment against the CNN-FPC model (As illustrated in FIG. 7B Diagram E, column 4).

For small and moderate offsets, the CNN-FPC corrected spectra and MLP-FPC corrected spectra (As illustrated in FIG. 7A Diagram B, columns 2-3) are similar to the original spectra (As illustrated in FIG. 7A Diagram B, column 1). However, for large offsets, the MLP-FPC corrected spectra (As illustrated in FIG. 7A Diagram B, column 4) slightly diverge from the original spectra, while the CNN-FPC corrected spectra still are not noticeably different from the original spectra. Comparably, the CNN-SR+ embodiment corrected spectra is consistent with the original spectra, regardless of the scale of offsets added. The superior performance of the CNN-SR+ embodiment was also indicated by the variances of choline interval for the 101 in vivo datasets (As illustrated in FIG. 8). With no offset or large offset, the CNN-FPC model had lower choline interval variance than the MLP-FPC model; but with small or medium offsets, the MLP-FPC model had lower choline interval variance than the CNN-FPC model. The CNN-SR+ embodiment, in contrast, had relatively stable performance and its generated variance of the choline interval was significantly lower than both the MLP-FPC model and the CNN-FPC model at all offset levels. Also, the larger the offset, the more significant were the CNN-SR+ embodiment results.

To demonstrate the robustness and spectra quality, tests were also performed to compare the best-performed CNN-SR+ embodiment and the published non-deep learning approach, model-based SR (mSR) [See Cited References: 3](As illustrated in FIG. 9). mSR exhibited the same performance pattern as the CNN-SR+ embodiment, with a similar mean performance score of 0.50±0.07 for small offsets. They had a similar level of variance of choline interval at around 0.6×10−4, with no significant difference.

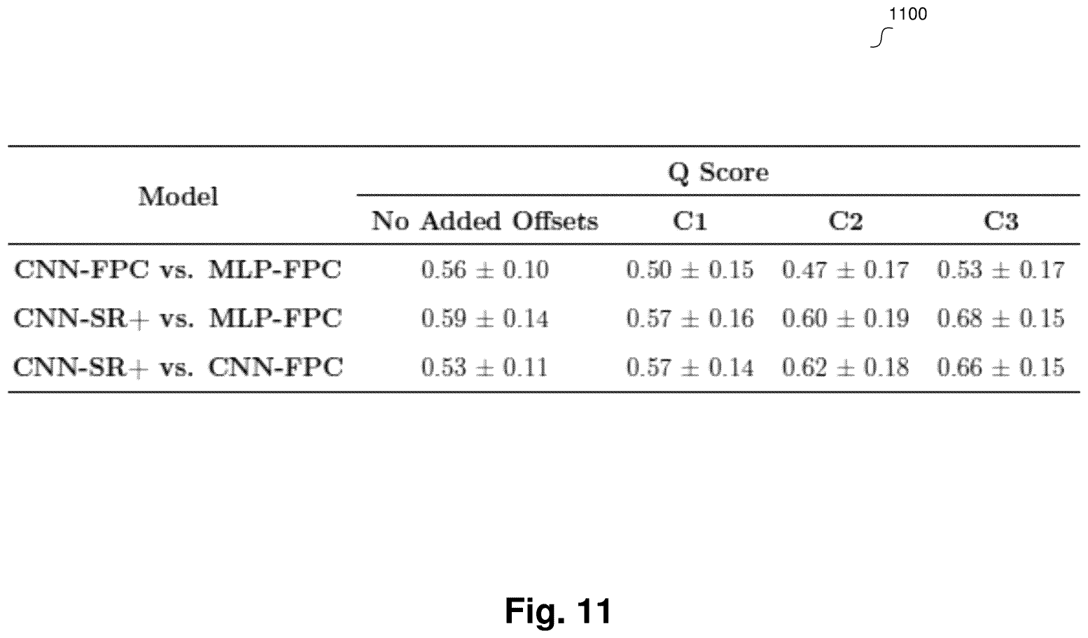

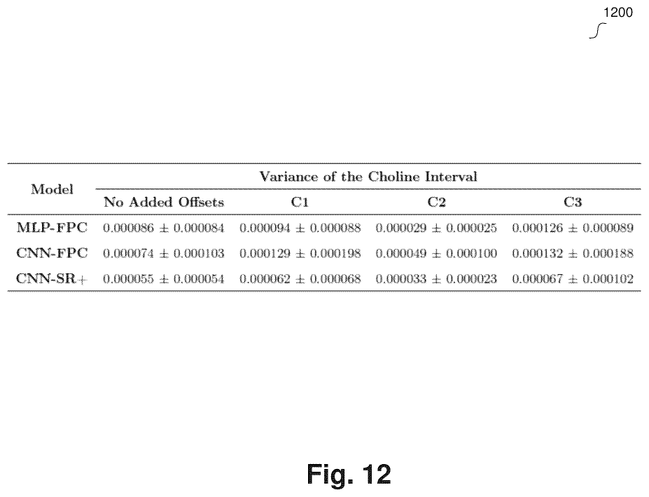

FIG. 10 illustrates table 1000 that contains mean absolute errors of the MLP-FPC model, CNN-FPC model, CNN-SR embodiment, and CNN-SR+ embodiment for frequency correction, phase correction, GABA residual and Glx residual on the simulation dataset with different levels of noise. FIG. 11 illustrates table 1100 of performance scores Q calculated between the MLP-FPC model, CNN-FPC model, and CNN-SR+ embodiment under four conditions: no added offsets, small offsets (C1), moderate offsets (C2) and large offsets. FIG. 12 illustrates table 1200 of choline residuals calculated on the MLP-FPC model, CNN-FPC model, and CNN-SR+ embodiment under four conditions: no added offsets, small offsets (C1), moderate offsets (C2) and large offsets (C3).

### Discussion of First Example Implementation of CNN-SR and CNN-SR+

The metabolic profile of both human and animal brains may be non-invasively and quantitatively measured using MRS. The MRS research and clinical applications have provided invaluable information on the metabolic state of the brain. However, MRS data collection and analysis can be improved, since MRS is prone to scanner instability brought on by factors like frequency drift and subject motion. In order to accurately represent and measure metabolites, FPC through spectral registration is a common preprocessing step that avoids unwanted spectral distortions which may bias the metabolite quantification.

From FIGS. 5, 4, 6A, and 6B, the results show that the proposed CNN-SR embodiment is more robust and has a better performance when compared to the other sequential FPC deep learning methods (CNN-FPC and MLP-FPC) for all testing conditions in the simulated data. FIGS. 4, 6A, and 6B demonstrate that in simulated data with SNR 20, the MLP-FPC model exhibits larger correction errors for frequency and phase offset, and On/OFF mismatch errors followed by the CNN-FPC model, with both being outperformed by the CNN-SR embodiment. The scatter plots also demonstrate that the CNN-SR embodiment offset predictions are more congregated near the y=0, indicating better prediction accurate than the FPC models. The single residual spectra that plots the difference between the prediction and the ground truth also exhibits consistent results, where a more complete subtraction (straight line) is obtained.

Likewise, FIGS. 5, 6A, and 6B confirm that the CNN-SR embodiment surpasses both sequential FPC deep learning methods when faced with more distorted data (SNR 2.5 with LB 0-20 ms). The results show that the CNN-SR embodiment had smaller mean absolute errors for both frequency and phase offsets predictions and Diff spectra derivation evidencing this approach is more robust to noise and provides more accurate predictions.

Moreover, the performance of the CNN-SR embodiment can be improved in some scenarios. Given the nature of in vivo data where ground truths are not present and the data is disturbed by multiple variables (e.g., noise, subject motion, etc.), an unsupervised learning spectral registration approach can further enhance the model performance. A refined model (CNN-SR+) was further trained in an embodiment with the more distorted data from the pre-trained CNN-SR embodiment. FIGS. 5, 6A, and 6B demonstrate that the CNN-SR+ embodiments achieved improved results as seen in the smaller correction errors for both phase and frequency, and in the subtraction of the prediction to the ground truth Diff spectra showing a straight line.

When testing on in vivo data for different phase and frequency offsets, the utility of an unsupervised spectral registration model, the CNN-SR+ embodiment, demonstrated once again to have high performance. FIGS. 7A and 7B show that OFF and Diff spectra are clearer for this embodiment across testing conditions, with shapes and peaks better preserved. Furthermore, Q scores are consistently higher in the CNN-SR+ embodiment in comparison to all the other FPC deep learning models tested.

Embodiments illustrate the value of deep learning for spectral registration and evince the utility and strengths of simultaneous FPC with a spectral registration model framework. The CNN-SR embodiment can perform simultaneous frequency-and-phase correction, and compared to the CNN-FPC and MLP-FPC models, it produces more reliable, robust and accurate results in a shorter processing time and with higher computational efficiency. Additionally, embodiments have the capability of adopting an unsupervised learning approach. Contrary to other FPC models that use a ground truth, this approach can take advantage of using spectra loss to learn in an unsupervised manner. This flexibility is a significant advantage, widely applicable to training and testing on in vivo data and other suitable data. It can also be shown that the performance of the CNN-SR+ embodiment is comparable to mSR, but given its advantages as stated previously (e.g., shorter processing time and higher computational efficiency), embodiments surpass the utility of mSR. Results of embodiments reveal that employing unsupervised learning to fine-tune models can achieve state of the art performance for any given dataset.

### Second Example Implementation of CNN-SR+: Data Sets

### Simulated Dataset

One challenge deep learning techniques often experience is determining the inputs and ground truth for model training, for example to achieve a specific performance goal. Since the ground truth of frequency and phase offsets for the in vivo dataset is not available, embodiments simulated the MEGA-PRESS training, validation, and test transients using an FID-A toolbox (version 1.2) in Matlab R2021a, (The Mathworks, Natick, Massachusetts, USA) with the same parameters (15-msec sinc-Gaussian editing pulses with FWHM=88.5 Hz, 2048 data points sampled at 2 kHz spectral width) as described in the previous work [See Cited References: 10,14]. The training set was allocated 32,000 OFF+ON spectra (2048 points each), and 4000 for both validation set and test set. Other suitable set breakdowns can be implemented. Embodiments were tested using datasets with added random Gaussian noise at SNR 20 and further challenged the model with lower SNR 2.5 and line broadening (0-20 msec). The SNR values were computed by the ratio of the Cr peak signal relative to the noise standard deviation.

### In Vivo Data Sets

In vivo data involved in some embodiments was retrieved from the publicly available Big GABA repository [See Cited References: 15]. In addition, 101 medial parietal lobe MEGA-edited datasets from nine sites with (Philips Healthcare, Best, The Netherlands) scanners (3 T field strength, TE=68 msec, 2048 data points sampled at 2 kHz spectral width, 320 transients) were collected in total, where each dataset contained 320 transients OFF+ON. The in vivo cohort data had the following guidelines: 18-35 years old; approximately 50:50 female/male split. The models were also evaluated on this in vivo dataset with additional offsets (small, medium, large) introduced.

### Second Example Implementation of CNN-SR+: Network Architecture

Embodiments incorporate both supervised and unsupervised learning in the proposed CNN-SR+ techniques on the simulation dataset. Some embodiments further fine tune this training using unsupervised learning on the in vivo dataset. During supervised training, both supervised loss and unsupervised loss were implemented to optimize the network parameters, as illustrated in FIG. 1. Based on the CNN-SR model, CNN-SR+ model used unsupervised loss for training related to the in vivo dataset.

FIG. 3B illustrates an example neural network according to some embodiments. Network 302 illustrates a sequential network that takes moving spectra and template spectra as inputs and predicts frequency and phase offsets at the same time (e.g., simultaneously). In some implementations, both moving spectra and template spectra are processed to have length of 1024 and are concatenated to form a single 2048 input array. Other suitable sizes, orientations, and dimensions can be implemented.

Network 302 starts with successive layers (e.g., four), each comprising a one dimensional convolutional layer followed by a batch-normalization layer and a one-dimensional max-pooling layer. The convolutional layer of network 302 comprises (By order: 2, 4, 8, 16) kernels with a size of 128, and the max-pooling layer comprises a pool size of 2 with a stride of 2. Network 302 includes three fully connected layers (FC) with 1024, 512, and 256 nodes and a final fully connected linear output layer of 2 nodes. Any other suitable fully connected layers can be implemented. In network 302, each hidden layer is followed by a rectified linear unit (ReLU) activation function to introduce non-linearity. Network 302 can be trained with an Adam optimizer, for example with a 0.0001 learning rate [See Cited References: 16]. The output from network 302 can be the predicted offset of frequency and phase. Network 302 can be trained for 1000 epochs with a batch size of 320, and the mean absolute error (MAE) can be used as the loss function. Any other suitable training parameters can be utilized.

### Network Training

In some implementations, MAE loss was used to compute the differences between the predicted and true offsets and spectra. At the training stage, the model's loss function can include two parts, supervised loss, and unsupervised loss (as illustrated in FIG. 1). The supervised loss computes the difference between the predicted and true frequency and phase offsets. The unsupervised loss computes the difference between the registered real and imaginary spectra from the predicted offsets with the template spectra. The loss functions were defined together to form a semi-supervised loss function shown as the following:

$$Loss = 100 \times ( RealSpectraMAE + ImaginarySpectraMAE ) + \frac{FrequencyMAE}{10} + \frac{PhaseMAE}{20} ,$$

With respect to the above loss function, weights and normalization factors can be implemented to optimize the training. Given more extreme or in vivo datasets where ground truths are difficult to obtain, embodiments can cope with this challenge by fine-tuning pre-trained model parameters using the unsupervised component of the loss function. This allows embodiments to adapt to the specific dataset and improve performance to achieve improved SR practice.

### Second Example Implementation of CNN-SR+: Evaluation and Comparison Using The In Vivo Dataset

The MEGA-edited datasets were used as the test set of the CNN-SR+ embodiment. For a first comparison to the performance of the CNN model, a published model-based SR (mSR), a non-deep learning approach, was used to perform FPC in the time domain [See Cited References: 3]. Specifically, mSR uses a noise-free model as the template instead of the median transient of the dataset. Noise-free ON and OFF FID models were created in Osprey (version 1.0.0), an open-source MatLab toolbox (The Mathworks, Natick, Massachusetts, USA), following previous preprocessing recommendations [See Cited References: 15]. The CNN-SR+ embodiment was also compared to a benchmark neural network MLP-FPC, using MLP containing 3 FC layers (1024, 512, 1 node(s)) and CNN-FPC, a CNN containing two convolutional blocks (Convolutional layer with 4 kernels of size 3 and Max pooling layer with down-sampling size 2 and stride 2) and 3 FC layers (1024, 512, 1 node(s)) [See Cited References 10,14]. In both of these networks, each hidden FC layer was followed by a ReLU activation function, and a linear activation function followed the output layer.

To examine the network in a more extreme environment, additional series of artificial offsets were added to the in vivo data. There were three different kinds of additionally added offsets: 1) 0≤|Δf|≤5 Hz and 0≤|Δϕ|≤20; 2) 5≤|Δf|≤10 Hz and 20≤|Δϕ|≤45; 3) 10≤|Δf|≤20 Hz and 45≤|Δϕ|≤90. Additional offsets were sampled from a uniform distribution and added as random pairs of frequency and phase to each transient.

### Second Example Implementation of CNN-SR+: Performance Measurement

In the simulated dataset, the artificial offsets were set as the ground truth, and the MAE between the ground truth and predicted value was used as the criteria to measure the network's performance. Moreover, calculation and plotting of the difference value between the true spectra and the corrected spectra using mSR, MLP-FPC, CNN-FPC, and CNN-SR+ was performed. A Q score was used to determine the performance strengths of each methods, and it was defined as Q=1−σ₁²/(σ₁²+σ₂²), where σ² is the variance of the choline subtracted artifact in the average difference spectrum [See Cited References: 14]. If the Q score was greater than 0.5, it indicated that the first method performed better than the second method and vice versa. The computation time of CNN-SR+ and mSR per transient was also measured.

### Second Example Implementation of CNN-SR+: Statistical Analysis

A two-tailed paired t-test was used to generate the P-value comparing CNN-SR+'s MAE to the other approaches' (MLP-FPC and CNN-FPC) MAE when testing on the simulated test set (SNR 20 and SNR 2.5 with line broadening). For each modality comparison (CNN-SR+ vs. MLP-FPC, and CNN-SR+ vs. CNN-FPC for SNR 20; CNN-SR+ vs. MLP-FPC, and CNN-SR+ vs. CNN-FPC for SNR 2.5 with line broadening), the statistical significance was determined. Moreover, a two-tailed paired t-test was used and the P-value of the variance of the choline interval was computed to determine the statistical significance of CNN-SR+ compared to the other approaches (MLP-FPC, CNN-FPC, and mSR) when using the in vivo dataset. A P-value <0.05 was considered statistically significant in both analyses.

### Second Example Implementation of CNN-SR+: Results

### Visual Comparison of Model Performance

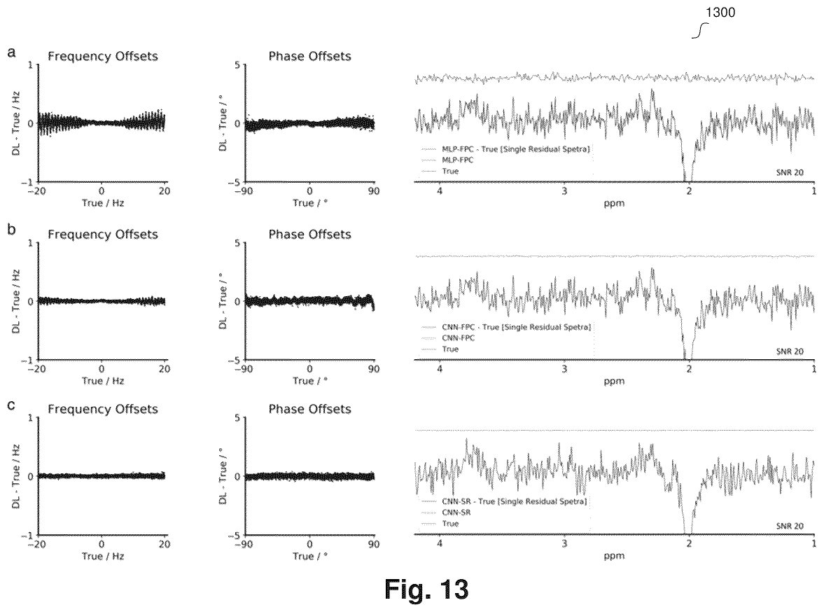

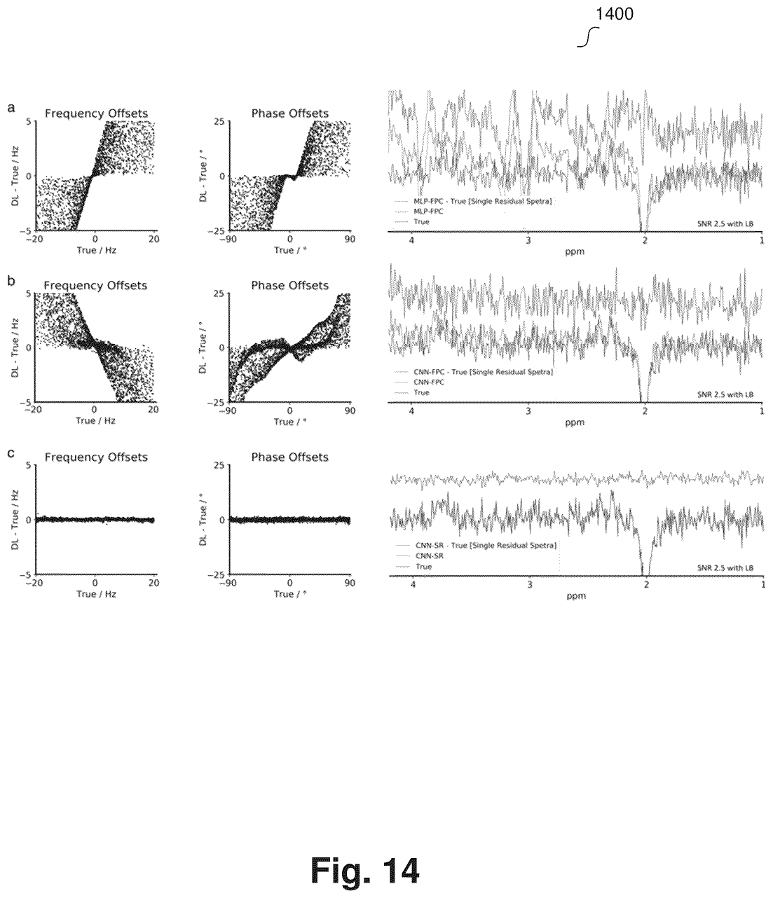

FIGS. 13 and 14 illustrate visualizations of the performance of the deep learning models for frequency-and-phase correction according to a second example implementation. Diagram 1300 illustrates a visualization of the performance of the deep learning models (MLP-FPC, CNN-FPC, CNN-SR+) for frequency and phase correction using the published simulated dataset with added noise at SNR of 20. For the MLP-FPC model, the CNN-FPC model, and the CNN-SR+ embodiment, the scatter plots on the left of diagram 1300 show the correction errors between the ground truths and model predictions at different frequency and phase offsets. The spectra on the right of diagram 1300 demonstrate the spectra predicted by each deep learning model, the true MEGA-PRESS difference spectra, and the subtraction between them. Among all three models, the MLP-FPC exhibits larger correction errors for frequency and phase offset followed by the CNN-FPC, with both being outperformed by the CNN-SR+ embodiment. Diagram 1300 illustrates: (a) Output of the MLP-FPC model on the simulated dataset; (b) Output of the CNN-FPC model on the simulated dataset; (c) Output of the CNN-SR+ embodiment on the simulated dataset.

Diagram 1400 of FIG. 14 illustrates a visualization of the performance of the deep learning models (MLP-FPC, CNN-FPC, CNN-SR+) for frequency and phase correction using the published simulated dataset with line broadening and added noise at SNR of 2.5. For the MLP-FPC model, the CNN-FPC model, and the CNN-SR+ embodiment, the scatter plots on the left of diagram 1400 show the correction errors between the ground truths and model predictions at different frequency and phase offsets. The spectra on the right of diagram 1400 demonstrate the spectra predicted by each deep learning model, the true MEGA-PRESS difference spectra, and the subtraction between them. Among all three models, MLP-FPC exhibits larger correction errors for frequency and phase offset followed by CNN-FPC, with all being outperformed by CNN-SR+. Diagram 1400 illustrates: (a) output of the MLP-FPC model on the simulated dataset; (b) output of the CNN-FPC model on the simulated dataset; (c) output of the CNN-SR+ embodiment on the simulated dataset.

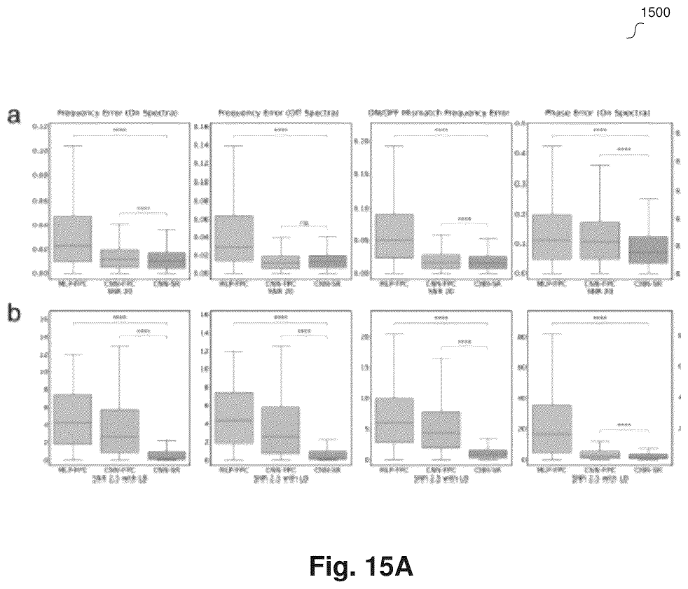

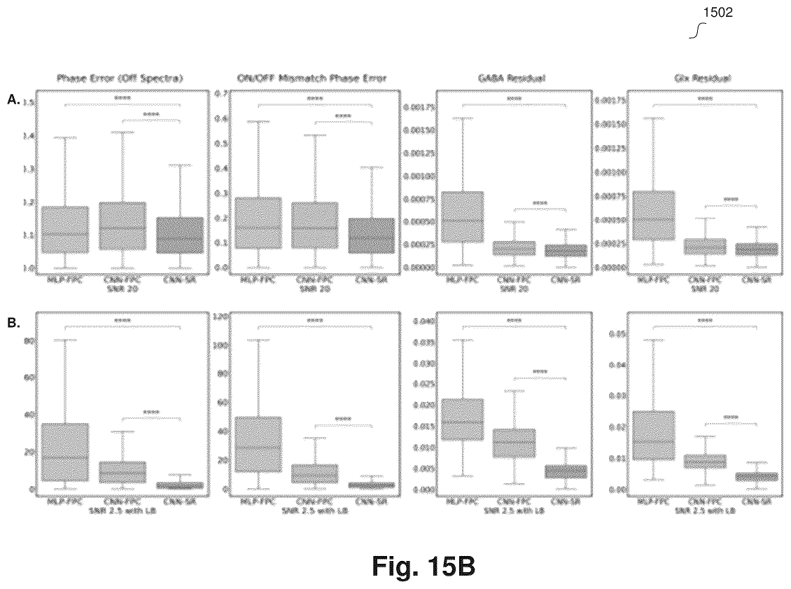

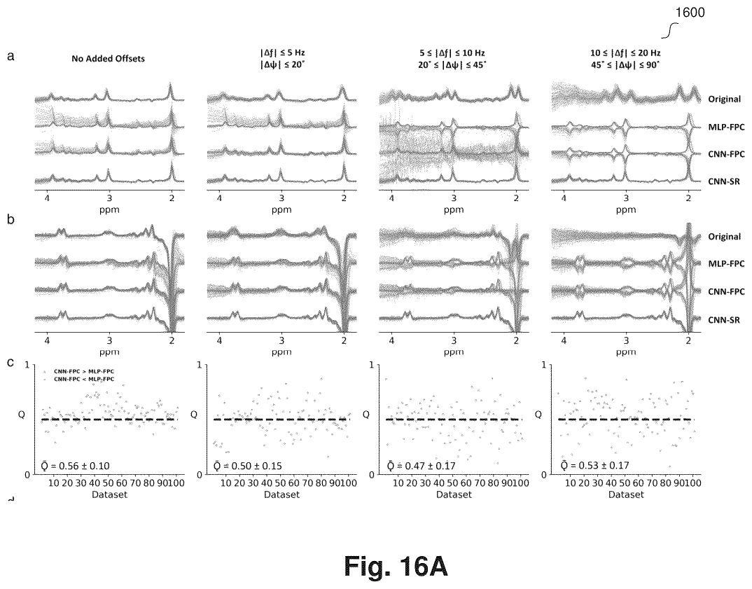

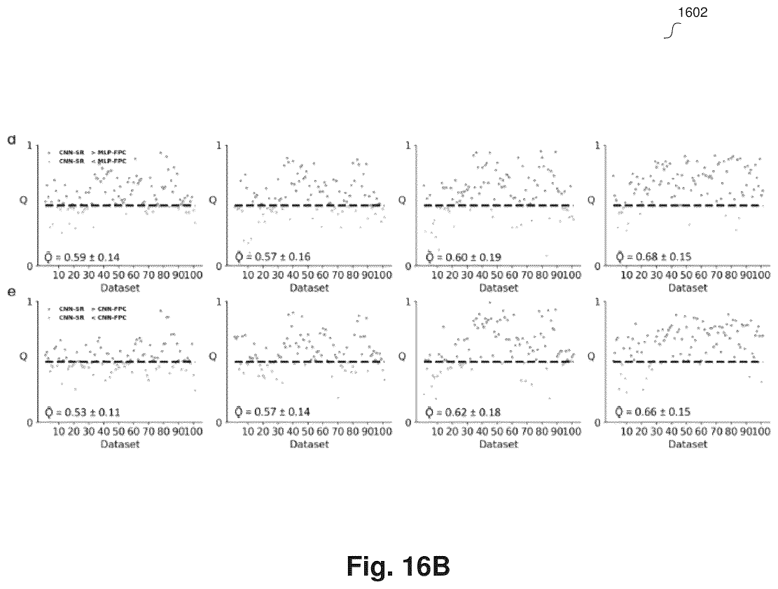

FIGS. 15A, 15B, 16A, and 16B illustrate visual comparison between deep learning models for frequency-and-phase correction of On, OFF and Diff spectra according to a second example implementation. Diagrams 1500 and 1502 illustrate a comparison between the MLP-FPC model, the CNN-FPC model and the CNN-SR+ embodiment for frequency-and-phase correction of the On spectra, Off spectra and On/Off mismatch at SNR of 20 and at SNR of 2.5 with line broadening. From left to right, diagrams 1500 and 1502 show: the frequency estimation error of the On spectra, the frequency estimation error of the Off spectra, the frequency On/Off mismatch error, the phase estimation error of the On spectra, the phase estimation error of the Off spectra, the phase On/Off mismatch error, the GABA residual spectra mean absolute error and the Glx residual spectra mean absolute error. Diagrams 1500 and 1502 include: (a) Box plots showing the frequency estimation errors (in Hz), the phase estimation errors (in degrees) and the GABA and the Glx residual spectra mean absolute error of the MLP-FPC model, the CNN-FPC model, and the CNN-SR+ embodiment at SNR of 20; (b) Box plots showing the frequency estimation errors (in Hz), the phase estimation errors (in degrees) and the GABA and the Glx residual spectra mean absolute error of the MLP-FPC model, the CNN-FPC model, and the CNN-SR+ embodiment at SNR 2.5 with line broadening. With respect to the symbols “****” in diagrams 1500 and 1502: The two-tailed P-value is less than 0.0001.

Diagrams 1600 and 1602 illustrate the in vivo Off and Diff spectra results of models with different levels of added offsets and performance scores comparing the CNN-FPC model to the MLP-FPC model, the CNN-SR+ embodiment to the MLP-FPC model, and the CNN-SR+ embodiment to the CNN-FPC model for the 101 in vivo datasets. Diagrams 1600 and 1602 include: (a) The original Off spectra and the results of three models after applying corrections to the in vivo data without further manipulation and with additional frequency and phase offsets applied to the same 101 datasets: small offsets (0-5 Hz; 0-20), medium offsets (5-10 Hz; 20-45), and large offsets (10-20 Hz; 45-90); (b) The original Diff spectra and the results of three models after applying corrections to the in vivo data without further manipulation and with additional frequency and phase offsets applied to the same 101 datasets: small offsets (0-5 Hz; 0-20), medium offsets (5-10 Hz; 20-45), and large offsets (10-20 Hz; 45-90); (c) Comparative performance Q scores for the CNN-FPC model and the MLP-FPC model for each dataset. A score above 0.5 indicated that the CNN-FPC model performed better than the MLP-FPC model in terms of alignment, whereas a score below 0.5 indicated the opposite. (d) Comparative performance Q scores for the CNN-SR+ embodiment and the MLP-FPC model for each dataset. (e) Comparative performance Q scores for the CNN-SR+ embodiment and the CNN-FPC model for each dataset.

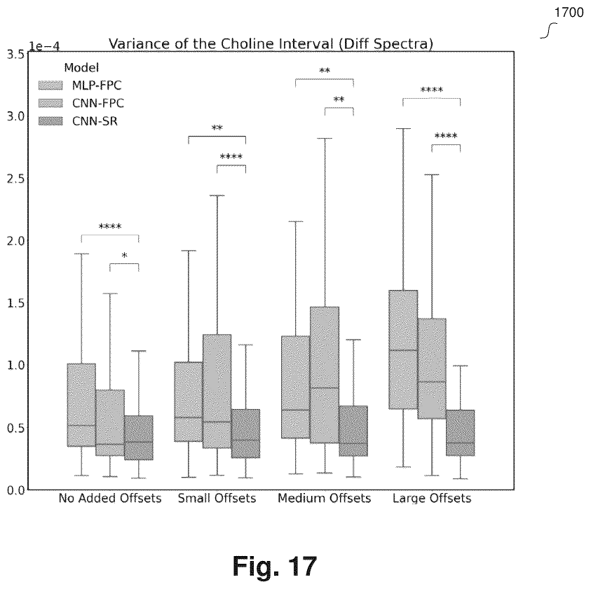

FIG. 17 illustrates a visual comparison of the variance of a choline interval in edited in vivo difference spectra among deep learning models with different levels of added offsets according to a second example implementation. Diagram 1700 illustrates a visual comparison of the variance of the choline interval in the edited in vivo Diff spectra among the MLP-FPC model, CNN-FPC model, and CNN-SR+ embodiment with different levels of added offsets. From left to right, diagram 1700 includes: box plots of choline interval variances with no offset, small offsets, medium offsets and large offsets. The CNN-SR+ embodiment has relatively stable performance and its generated variance of the choline interval is significantly lower than both the MLP-FPC model and the CNN-FPC model at all offset levels. With no offset or large offset, the CNN-FPC model has lower choline interval variance than the MLP-FPC model; but with small or medium offsets, the MLP-FPC model has lower choline interval variance than the CNN-FPC model. With respect to the following symbols in diagram 1700: “****”—The two-tailed p-value is less than 0.0001; “**”—The two-tailed p-value is between 0.001 and 0.01; “*”—The two-tailed p-value is between 0.01 and 0.05.

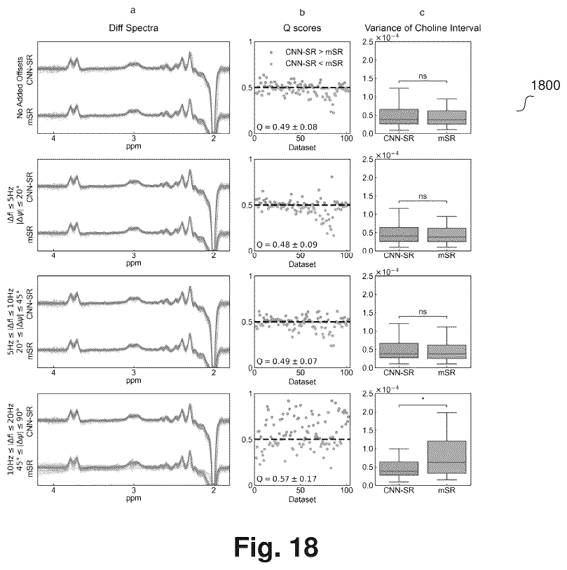

FIG. 18 illustrates a visual comparison of model performance for an embodiment of the convolutional neural network based spectral registration model and a numerical method model-based spectral registration for in vivo datasets, in terms of the Off spectra, Diff spectra, performance scores, and variance of choline interval, according to a second example implementation. Diagram 1800 illustrates model performance comparison between the CNN-SR+ embodiment and mSR model for the in vivo datasets, in terms of the Off spectra, Diff spectra, performance scores and the variance of choline interval for without and with additional offsets. Diagram 1800 includes: (a) The Diff spectra results of the CNN-SR+ embodiment and the mSR model. (b) Comparative performance scores Q for the CNN-SR+ embodiment and the mSR model for each dataset. A score above 0.5 indicated that the CNN-SR+ embodiment performed better than the mSR model in terms of alignment, whereas a score below 0.5 indicated the opposite. (c) Box plots of the variance of the choline interval of the CNN-SR+ embodiment and the mSR model. No significant difference is observed in the no, small and medium additional offset cases but a significant difference is observed in the large additional offset case.

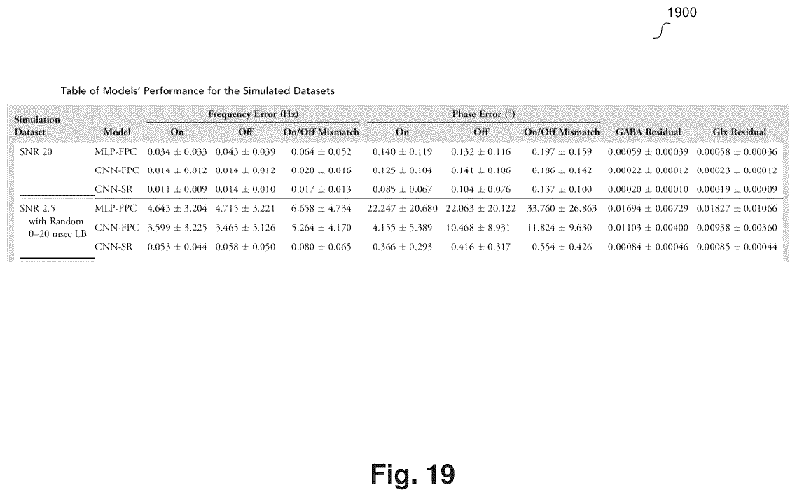

FIG. 19 illustrates a table of mean absolute errors of deep learning model performance for frequency correction, phase correction, GABA residual, and Glx residual on the simulation dataset with different levels of noise according to a second example implementation. Table 1900 includes mean absolute errors of the MLP-FPC model, CNN-FPC model, and CNN-SR+ embodiment for frequency correction, phase correction, GABA residual, and Glx residual on the simulation dataset with different levels of noise.

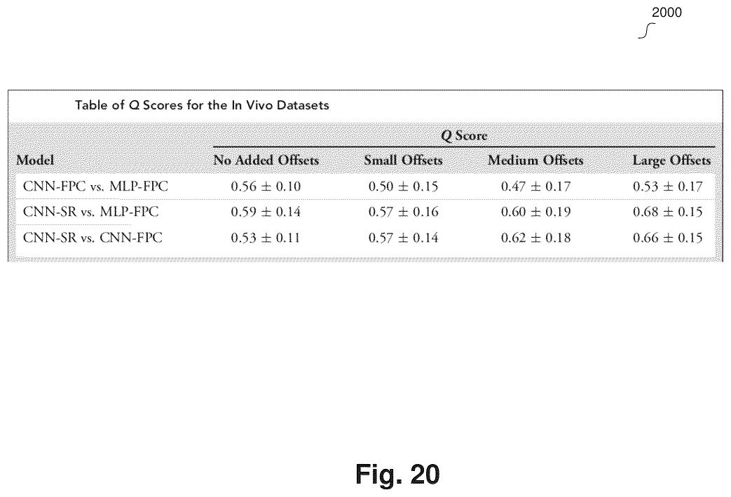

FIG. 20 illustrates a table of model performance scores Q calculated between deep learning models under varying conditions according to a second example implementation. Table 2000 includes performance scores Q calculated between MLP-FPC model, CNN-FPC model, and CNN-SR+ embodiment under four conditions: no added offsets, small offsets, medium offsets, and large offsets.

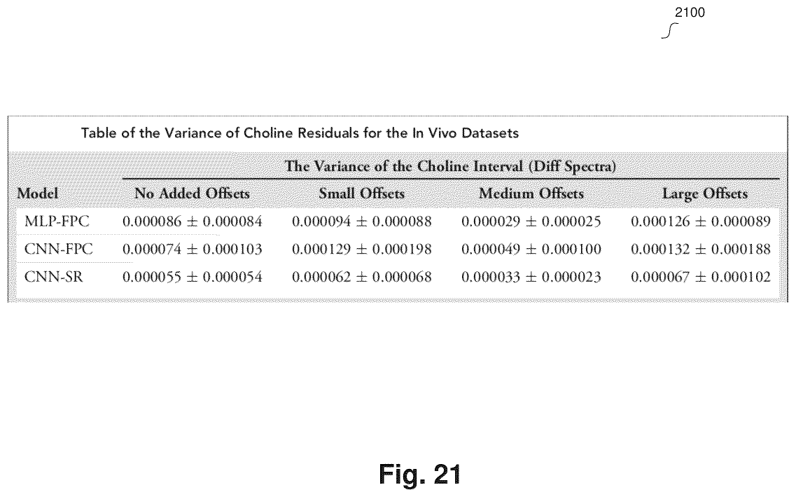

FIG. 21 illustrates a table of choline variances calculated using deep learning models under varying conditions according to a second example implementation. Table 2100 includes choline variances calculated on the MLP-FPC model, CNN-FPC model, and CNN-SR+ embodiment under four conditions: no added offsets, small offsets, medium offsets, and large offsets.

### Model Performance Evaluation and Spectra Analysis for the Simulated Datasets

The results of the MLP-based approach and CNN-based approaches on the simulated test dataset with SNR of 20 and SNR of 2.5 with line broadening are illustrated in FIGS. 13 and 14. The comparison of the errors for FPC of the MLP-based approach and the CNN-based approaches of the On spectra, Off spectra, and On/Off mismatch of the simulated test set for varying SNRs is illustrated in FIGS. 15A and 15B.

For the test set with SNR 20, the CNN-based approaches showed significantly lower frequency estimation errors than the MLP-based approach, and the CNN-SR+ embodiment showed the lowest phase estimation errors for the On spectra, Off spectra and On/Off mismatch (as illustrated in FIGS. 15A and 15B, Diagram A). Taking the Off spectra as an example, the mean frequency offset errors were 0.043±0.039 Hz for the MLP-FPC model, 0.014±0.012 Hz for the CNN-FPC model, and 0.014±0.010 Hz for the CNN-SR+ embodiment. The mean phase offset errors were 0.132±0.116 for the MLP-FPC model, 0.141±0.106 for the CNN-FPC model, and 0.104±0.076 for the CNN-SR+ embodiment.

With a lower SNR of 2.5 with random 0-20 msec line broadening introduced (as illustrated in FIGS. 15A-15B, Diagram B), the CNN-SR+ embodiment demonstrated significantly lower frequency and phase estimation errors than the other models for the On spectra, Off spectra, and On/Off Mismatch. For example, the mean frequency and phase offset estimation errors for the Off spectra were 4.715±3.221 Hz and 22.063±20.122 for the MLP-FPC model, 3.465±3.126 Hz and 10.468±8.931 for the CNN-FPC model, and 0.058±0.050 and 0.416±0.317 for the CNN-SR+ embodiment.

The results in FIGS. 15A, 15B, 16A, and 16B show that compared to the MLP-based approach, the CNN-based approaches had smaller errors within the frequency and phase ranges tested. At SNR of 20, the CNN-SR+ embodiment performed better than the MLP-FPC model and the CNN-FPC model. When the SNR decreased to 2.5 and line broadening was applied, the CNN-SR+ embodiment performed better than the MLP-FPC and CNN-FPC models, which had less stable predictions and larger errors.

Additionally, by extracting the spectra interval corresponding to GABA (i.e., 2.8-3.2 ppm) and Glx (i.e., 3.55-3.95 ppm) from the derived mean difference spectra (As illustrated in FIGS. 15A and 15B), the residual spectra errors were found to be lower with the CNN-SR+ embodiment (at SNR of 20 and SNR of 2.5 with line broadening) than with the MLP-FPC and CNN-FPC models. Consequently, the residual spectra errors using CNN-based models for the full spectra were lower than those of the MLP-based model for the On spectra, Off spectra, and On/Off mismatch at a lower SNR, indicating CNN-based models' higher performance and robustness in the presence of noise with respect to the MLP-FPC model. Among CNN-based models, the CNN-SR+ embodiment performed best in terms of frequency and phase estimation errors and noise tolerance (numerical results are shown in table 1900 of FIG. 19). Results were statistically significant.

### Model Performance Evaluation and Spectra Analysis for the In Vivo Big GABA Datasets

FIG. 16A, Diagrams A and B illustrate the Off and Diff spectra resulting from the 101 in vivo Big GABA datasets without (column 1) or with (columns 2-4) additional artificial offsets for no correction MLP-FPC model correction, CNN-FPC model correction, and CNN-SR+ embodiment correction. The additional frequency and phase offsets applied to the same 101 datasets were small offsets (0-5 Hz; 0-20), medium offsets (5-10 Hz; 20-45), and large offsets (10-20 Hz; 45-90). FIG. 16A diagram C and FIG. 16B diagrams D and E demonstrate performance scores comparing the CNN-FPC model to the MLP-FPC model, the CNN-SR+ embodiment to the MLP-FPC model, and the CNN-SR+ embodiment to the CNN-FPC model for the 101 in vivo datasets.

When small offsets were added, the MLP-FPC and CNN-FPC models performed similarly (mean performance score 0.50±0.15; FIG. 5c, column 2), and both were outperformed by the CNN-SR+ embodiment. The mean performance score of the CNN-SR+ embodiment against the MLP-FPC model score of the CNN-SR+ embodiment against the MLP-FPC model was 0.57±0.16 (FIG. 16B, diagram D, column 2), and it was 0.57±0.14 for the CNN-SR+ embodiment against the CNN-FPC model (FIG. 16B, diagram E, column 2).

As for medium offsets, the performance of the MLP-FPC model and the CNN-FPC model was comparable, but the CNN-SR+ embodiment still performed better. The mean performance score of the CNN-FPC model against the MLP-FPC model was 0.47±0.17 (FIG. 16A, diagram C, column 3), while it was 0.60±0.19 for the CNN-SR+ embodiment against the MLP-FPC model (FIG. 16B, diagram D, column 3), and 0.62±0.18 for the CNN-SR+ embodiment against the CNN-FPC model (FIG. 16B, diagram E, column 3).

When large offsets were added, the performance of the CNN-FPC model was slightly better than the MLP-FPC model. The CNN-SR+ embodiment still outperformed the MLP-FPC and CNN-FPC models. The mean performance score of the CNN-FPC model against the MLP-FPC model was 0.53±0.17 (FIG. 16A, diagram C, column 4), while it was 0.68±0.15 for the CNN-SR+ embodiment against the MLP-FPC model (FIG. 16B, diagram D, column 4), and 0.66±0.15 for the CNN-SR+ embodiment against the CNN-FPC model (FIG. 16B, diagram E, column 4).

For small and medium offsets, the CNN-FPC corrected spectra and MLP-FPC corrected spectra (FIG. 16A, diagram B, columns 2-3) were similar to the original spectra (FIG. 16A, diagram B, column 1). However, for large offsets, the MLP-FPC corrected spectra (FIG. 16A, diagram B, column 4) slightly diverged from the original spectra, while the CNN-FPC corrected spectra still had many consistencies in shape and size from the original spectra. Comparably, the CNN-SR+ embodiment corrected spectra was consistent with the original spectra, regardless of the scale of offsets added. The superior performance of the CNN-SR+ embodiment was also indicated by the variances of choline intervals for the 101 in vivo datasets (Diagram 1700 of FIG. 17). With no offset or large offset, the CNN-FPC model had lower choline interval variance than the MLP-FPC model; but with small or medium offsets, the MLP-FPC model had lower choline interval variance than the CNN-FPC model. The CNN-SR+ embodiment, in contrast, had relatively stable performance and its generated variance of the choline interval was significantly lower than both the MLP-FPC model and the CNN-FPC model at all offset levels. Also, the larger the offset, CNN-SR+ embodiment demonstrated superior performance compared to the MLP-FPC and the CNN-FPC. The results were statistically significant (See Tables 2000 and 2100 of FIGS. 20 and 21).

By comparing the best-performed CNN-SR+ embodiment to the published non-deep learning approach, mSR exhibited a similar performance pattern as the CNN-SR+ embodiment, with a similar mean performance score of 0.49±0.08 for no additional offsets (Diagram 1800 of FIG. 18). The same conclusion was drawn with small and medium additional offsets with a similar mean performance score of 0.48±0.09 and 0.49±0.07, respectively.

They had a similar level of variance of choline interval at around 0.6×10−4, with no significant difference. The P-values for no added offsets, small offsets, and medium offsets were 0.63, 0.41, and 0.20 respectively. For an input with large added offsets, the CNN-SR+ embodiment demonstrated significant improvement compared with mSR (0.57±0.17, P<0.05) which indicated the robustness of the CNN-SR+ embodiment to various input artifacts. The SR computation time of each transient given the in vivo dataset was also analyzed, where mSR had a processing time of 0.1475 s/transient while the CNN-SR+ embodiment had a processing time of 0.0415 s/transient.

### Discussion of Second Example Implementation of CNN-SR+

The metabolic profile of both human and animal brains may be non-invasively and quantitatively measured using MRS. It is beneficial for research and clinical applications since it provides information on the metabolic state of the brain. However, the collected data could be affected, since MRS is prone to scanner instability introduced by factors like frequency drift and subject motion. In order to accurately represent and measure metabolites, FPC through SR is often performed as a preprocessing step that avoids unwanted spectral distortions that may bias the metabolite quantification.

From the results, the CNN-SR+ embodiment was more robust and had superior performance when compared to other sequential FPC deep learning methods (CNN-FPC and MLP-FPC) for testing conditions in the simulated data. At SNR 20, the MLP-FPC model exhibited larger correction errors for frequency and phase offset, and On/Off mismatch followed by the CNN-FPC model, with both being outperformed by the CNN-SR+ embodiment. Likewise, the CNN-SR+ embodiment surpassed both sequential FPC deep learning methods when faced with more distorted data (SNR 2.5 with line broadening 0-20 msec). The results may show that CNN-SR+ had smaller MAEs for both frequency and phase offset predictions and Diff spectra derivation, thus this approach could be more robust to noise and provide more accurate predictions.

Moreover, due to the CNN-SR+ embodiment's unsupervised learning component, further fine-tuning the model to specific data is possible. Given the nature of in vivo data where ground truths are not present and the data is disturbed by multiple variables (i.e., noise, subject motion), an unsupervised learning SR approach can be used to further refine the model hyperparameters. By using CNN-SR+'s unsupervised learning framework, the pre-trained model was further trained with more distorted data (at SNR 2.5 with line broadening). It is clear how the CNN-SR+ embodiment can outperform the other FPC models, as seen in the smaller correction errors for both phase and frequency, and the residual spectra being smaller.

When testing on in vivo data for different phase and frequency offsets, the CNN-SR+ embodiment demonstrated once again to have superior performance. The Off and Diff spectra were clearer for this model across all testing conditions, with shapes and peaks better preserved. Furthermore, Q scores were consistently higher using the CNN-SR+ embodiment in comparison to all the other FPC deep learning models. Nevertheless, these results remain similar to mSR, the state-of-the-art non-deep learning numerical correction method, with no, small and medium additional offsets but were found to perform better when larger magnitudes of offsets were introduced [See Cited References: 3].

These findings illustrate the value of deep learning for SR and evince the utility and strengths of simultaneous FPC with a SR model framework. The CNN-SR+ embodiment can perform simultaneous frequency and phase correction and compared to the CNN-FPC and MLP-FPC models, it may produce more reliable, robust and accurate results in a shorter processing time and with higher computational efficiency. Additionally, the framework has the capability of adopting an unsupervised learning approach. Contrary to other FPC models that utilize a ground truth, this approach can take advantage of using spectra loss to learn in an unsupervised manner. This can be an advantage, widely applicable to training and testing on in vivo data. Additionally, the performance of CNN-SR+ was comparable to mSR when smaller/medium magnitudes of offsets exist in this dataset but given advantages as stated previously (shorter processing time and higher computational efficiency), CNN-SR+ surpasses the utility of mSR especially when larger offsets are introduced in the dataset. Results revealed that by employing unsupervised learning, fine-tuning the model to state-of-the-art performance for any given dataset can be performed.

The utility demonstrated by embodiments presents the opportunity for additional analysis. For example, embodiments of this disclosure were conducted using data from humans, but MRS is a widely available approach for animals as well, playing a noteworthy role in pre-clinical studies. Additionally, testing in the context of living conditions other than in vivo, such as in situ, ex vivo, and in vitro may further demonstrate the utility of embodiments.

Regarding the JDE sequences, sequences other than MEGA-PRESS, such as PRESS, sLASER, or MEGA-sLASER could be considered in the future to further demonstrate the utility of embodiments. Furthermore, data from other sources that are publicly available (e.g., General Electric and/or Siemens) can further demonstrate the utility of embodiments. Different magnetic field strengths other than 3 T (e.g., 9 T, 12 T) can also be further considered. Inclusion of other parameters, like first-order phase, amplitude, and bandwidth variance in different transients could also be further considered.

Although the CNN-SR embodiments outperformed the other deep-learning approaches, some results are still comparable to the state of the art model (mSR). However, given the advantages of embodiments when conducting spectral registration, such as high computational efficiency, quick processing time, and being able to generalize well on datasets from different modalities, embodiments of the CNN-SR techniques have more utility for users. Embodiments can also adopt to small datasets and can be applied to the same dataset numerous times, which can overcome potential issues such as lack of resources.

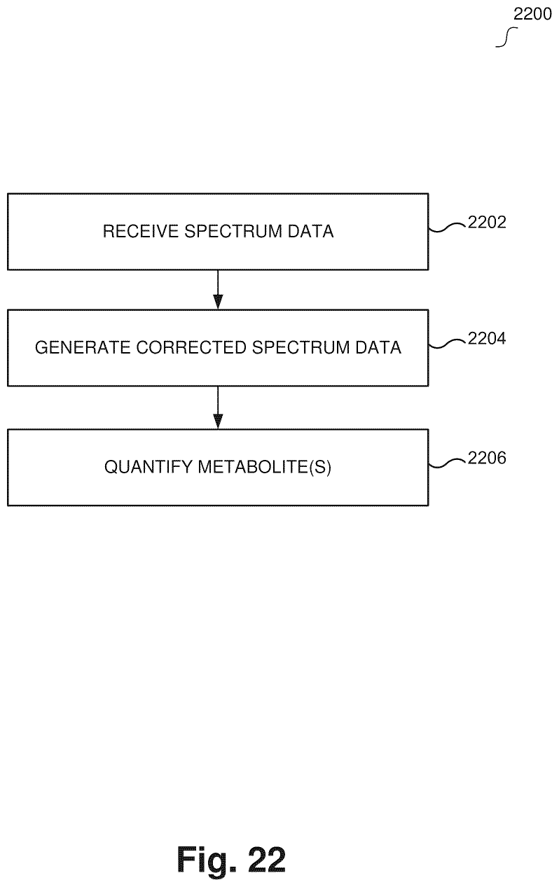

FIG. 22 illustrates a flow diagram for performing frequency-and-phase correction of magnetic resonance spectroscopy data to quantify one or more metabolites according to example embodiment. In one embodiment, the functionality of FIG. 22 is implemented by software stored in memory or other computer-readable or tangible medium, and executed by a processor. In other embodiments, each functionality may be performed by hardware (e.g., through the use of an application specific integrated circuit (“ASIC”), a programmable gate array (“PGA”), a field programmable gate array (“FPGA”), etc.), or any combination of hardware and software.

At block 2202, process 2200 can receive spectrum data generated using magnetic resonance spectroscopy. The spectrum data can relate to a subject's brain and a plurality of metabolite levels. For example, the received spectrum data can comprise single voxel MEGA-PRESS MRS data.

At block 2204, process 2200 can generate corrected spectrum data by inputting the received spectrum data to a trained convolutional neural network. The trained convolutional neural network can simultaneously estimate frequency corrections and phase corrections for the input spectrum data. In some implementations, the trained convolutional neural network comprises a single trained convolutional neural network, and, during training, the single convolutional neural network is trained to simultaneously estimate frequency corrections and phase corrections for spectrum training data. In some implementations, the trained convolutional neural network comprises a single trained convolutional neural network, and, during training, a loss function is used to train the single convolutional neural network using calculated loss based on both estimated frequency loss and estimated phase loss.

In some implementations, the convolutional neural network is trained using a first training phase and a second training phase. For example, the training data used to train the convolutional neural network comprises a simulation spectrum dataset and an in vivo spectrum dataset. The first training phase can train the convolutional neural network using the simulation spectrum dataset and the second training phase can train the convolutional neural network using the in vivo spectrum dataset. The first training phase can include a) supervised training, or b) supervised training and unsupervised training. The second training phase can include unsupervised training. In some implementations, the simulation spectrum dataset used for the first training phase includes training data labels for supervised training, and the in vivo spectrum dataset used for the second training phase does not include training data labels. In some implementations, the first training phase is performed prior to the second training phase.

At block 2206, process 2200 an quantify one or more metabolites using the corrected spectrum data. For example, the corrected spectrum data can be used to quantify metabolites using a MEGA-PRESS sequence. In some implementations, the quantified metabolite(s) comprise GABA, glutamate, or glutamine.

Embodiments demonstrate the utility of a CNN framework for MRS spectra registration with both supervised and unsupervised learning. Embodiments of the CNN-SR model show better performance and deliver results more robust to noise as compared to other state-of-the-art models contemplated in this disclosure in both simulation and in vivo tests.

The features, structures, or characteristics of the disclosure described throughout this specification may be combined in any suitable manner in one or more embodiments. For example, the usage of “one embodiment,” “some embodiments,” “certain embodiment,” “certain embodiments,” or other similar language, throughout this specification refers to the fact that a particular feature, structure, or characteristic described in connection with the embodiment may be included in at least one embodiment of the present disclosure. Thus, appearances of the phrases “one embodiment,” “some embodiments,” “a certain embodiment,” “certain embodiments,” or other similar language, throughout this specification do not necessarily all refer to the same group of embodiments, and the described features, structures, or characteristics may be combined in any suitable manner in one or more embodiments.

Reference in this specification to “implementations” (e.g., “some implementations,” “various implementations,” “one implementation,” “an implementation,” etc.) means that a particular feature, structure, or characteristic described in connection with the implementation is included in at least one implementation of the disclosure. The appearances of these phrases in various places in the specification are not necessarily all referring to the same implementation, nor are separate or alternative implementations mutually exclusive of other implementations. Moreover, various features are described which may be exhibited by some implementations and not by others. Similarly, various requirements are described which may be requirements for some implementations but not for other implementations.

As used herein, being above a threshold means that a value for an item under comparison is above a specified other value, that an item under comparison is among a certain specified number of items with the largest value, or that an item under comparison has a value within a specified top percentage value. As used herein, being below a threshold means that a value for an item under comparison is below a specified other value, that an item under comparison is among a certain specified number of items with the smallest value, or that an item under comparison has a value within a specified bottom percentage value. As used herein, being within a threshold means that a value for an item under comparison is between two specified other values, that an item under comparison is among a middle-specified number of items, or that an item under comparison has a value within a middle-specified percentage range. Relative terms, such as high or unimportant, when not otherwise defined, can be understood as assigning a value and determining how that value compares to an established threshold. For example, the phrase “selecting a fast connection” can be understood to mean selecting a connection that has a value assigned corresponding to its connection speed that is above a threshold.

As used herein, the word “or” refers to any possible permutation of a set of items. For example, the phrase “A, B, or C” refers to at least one of A, B, C, or any combination thereof, such as any of: A; B; C; A and B; A and C; B and C; A, B, and C; or multiple of any item such as A and A; B, B, and C; A, A, B, C, and C; etc.

Although the subject matter has been described in language specific to structural features and/or methodological acts, it is to be understood that the subject matter defined in the appended claims is not necessarily limited to the specific features or acts described above. Specific embodiments and implementations have been described herein for purposes of illustration, but various modifications can be made without deviating from the scope of the embodiments and implementations. The specific features and acts described above are disclosed as example forms of implementing the claims that follow. Accordingly, the embodiments and implementations are not limited except as by the appended claims.

One having ordinary skill in the art will readily understand that the embodiments as discussed above may be practiced with steps in a different order, and/or with elements in configurations that are different than those which are disclosed. Therefore, although this disclosure considers the outlined embodiments, it would be apparent to those of skill in the art that certain modifications, variations, and alternative constructions would be apparent, while remaining within the spirit and scope of this disclosure.

### CITED REFERENCES

1. Buonocore M H, Maddock R J. Magnetic resonance spectroscopy of the brain: A review of physical principles and technical methods. Rev Neurosci 2015; 26(6):609-632. https://doi.org/10.1515/revneuro-2015-0010 PMID: 26200810.
2. Mullins P G, McGonigle D J, O'Gorman R L, et al. Current practice in the use of MEGA-PRESS spectroscopy for the detection of GABA. Neuroimage 2014; 86:43-52.
3. Near J, Edden R, Evans C J, Paquin R, Harris A, Jezzard P. Frequency and phase drift correction of magnetic resonance spectroscopy data by spectral registration in the time domain. Magn Reson Med. 2015; 73:44-50.
4. Oeltzschner G, Zöllner H J, Hui S C N, et al. Open-source processing, reconstruction and estimation of magnetic resonance spectroscopy data. J Neurosci Methods. 2020; 343:108827.
5. Liu C, Ma D. J., et al (2021) JET—A Matlab toolkit for automated J-difference-edited M R spectra processing of in vivo mouse MEGA-PRESS study at 9.4 T. In Joint Annual Meeting ISMRM & SMRT 2021 (Vancouver, Canada).
6. Rothman D L, Petroff O A, Behar K L, Mattson R H. Localized 1H NMR measurements of gamma-aminobutyric acid in human brain in vivo. Proc Natl Acad Sci USA 1993; 90(12):5662-5666.
7. Guo J, Gang Z, Sun Y, Laine A, Small S A, Rothman D L. In vivo detection and automatic analysis of GABA in the mouse brain with MEGA-PRESS at 9.4 T. NMR Biomed 2018; 31:e3837. https://doi.org/10.1002/nbm.3837.
8. Mescher M, Merkle H, Kirsch J, Garwood M, Gruetter R. Simultaneous in vivo spectral editing and water suppression. NMR Biomed 1998; 11(6):266-72.
9. Sawiak S J, Jupp B, Taylor T, Caprioli D, Carpenter T A, Dailey J W. in vivo γ-aminobutyric acid measurement in rats with spectral editing at 4.7 T. J Magn Reson Imaging 2016; 43(6):1308-1312.
10. Ma D J, Le H A, Ye Y, et al. M R spectroscopy frequency and phase correction using convolutional neural networks. Magn Reson Med 2022; 87(4):1700-1710. https://doi.org/10.1002/mrm.29103.
11. de Graaf R A, Rothman D L. Spectral editing. eMagRes 2016; 5:1147-1156.
12. Fu Y, Lei Y, Wang T, et al. LungRegNet: An unsupervised deformable image registration method for 4D-CT lung. Med Phys 2020; 47(4):1763-1774. https://doi.org/10.1002/mp.14065.
13. Chen J, Frey E C, Du Y. Unsupervised learning of diffeomorphic image registration via TransMorph. In: Hering A, Schnabel J, Zhang M, Ferrante E, Heinrich M,
Rueckert D, editors. Biomedical Image Registration. WBIR 2022. Lecture Notes in Computer Science, Vol 13386. Cham: Springer; 2022.
14. Tapper S, Mikkelsen M, Dewey B E, Zöllner H J, Hui S C N, Oeltzschner G, Edden R A E. Frequency and phase correction of J-difference edited M R spectra using deep learning. Magn Reson Med. 2021 April; 85(4):I 755-1765. doi: 10.1002/mrm.28525. Epub 2020 Nov. 18. PMID: 33210342.
15. Mikkelsen M, Barker P B, Bhattacharyya P K, et al. Big GABA: Edited M R spectroscopy at 24 research sites. Neuroimage. 2017; 159:32-45.
16. D. Kingma and J. Ba. Adam: A method for stochastic optimization”. arXiv 2014, arXiv:1412.6980.
17. Brigham E O, Morrow R E. The fast fourier transform. IEEE Spectrum 1967; 4:63-70.

We claim:

1. A method for performing frequency-and-phase correction of magnetic resonance spectroscopy (MRS) data to quantify one or more metabolites, the method comprising:
receiving spectrum data generated using magnetic resonance spectroscopy, wherein the spectrum data relates to a subject's brain and a plurality of metabolite levels;
generating corrected spectrum data by inputting the received spectrum data to a trained convolutional neural network, wherein the trained convolutional neural network simultaneously estimates frequency corrections and phase corrections for the input spectrum data; and
quantifying one or more metabolites using the corrected spectrum data.

2. The method of claim 1, wherein the convolutional neural network is trained using a first training phase and a second training phase.

3. The method of claim 2, wherein the training data used to train the convolutional neural network comprises a simulation spectrum dataset and an in vivo spectrum dataset.

4. The method of claim 3, wherein the first training phase trains the convolutional neural network using the simulation spectrum dataset and the second training phase trains the convolutional neural network using the in vivo spectrum dataset.

5. The method of claim 4, wherein the first training phase is performed prior to the second training phase.

6. The method of claim 4, wherein,
the first training phase comprises a) supervised training or b) supervised training and unsupervised training, and
the second training phase comprises unsupervised training.

7. The method of claim 6, wherein
the simulation spectrum dataset used for the first training phase includes training data labels for supervised training, and
the in vivo spectrum dataset used for the second training phase does not include training data labels.

8. The method of claim 1, wherein the received spectrum data comprises single voxel MEGA-PRESS MRS data.

9. The method of claim 1, wherein the quantified one or more metabolites comprise GABA, glutamate, or glutamine.

10. The method of claim 1, wherein the trained convolutional neural network comprises a single trained convolutional neural network, and, during training, the single convolutional neural network is trained to simultaneously estimate frequency corrections and phase corrections for spectrum training data.

11. The method of claim 1, wherein the trained convolutional neural network comprises a single trained convolutional neural network, and, during training, a loss function is used to train the single convolutional neural network using calculated loss based on both estimated frequency loss and estimated phase loss.

12. A non-transitory computer readable medium having instructions stored thereon that, when executed by a processor, cause the processor to perform frequency-and-phase correction of magnetic resonance spectroscopy (MRS) data to quantify one or more metabolites, wherein the processor is configured to:
receive spectrum data generated using magnetic resonance spectroscopy, wherein the spectrum data relates to a subject's brain and a plurality of metabolite levels;
generate corrected spectrum data by inputting the received spectrum data to a trained convolutional neural network, wherein the trained convolutional neural network simultaneously estimates frequency corrections and phase corrections for the input spectrum data; and
quantify one or more metabolites using the corrected spectrum data.

13. The non-transitory computer readable medium of claim 12, wherein the convolutional neural network is trained using a first training phase and a second training phase.

14. The non-transitory computer readable medium of claim 12, wherein the training data used to train the convolutional neural network comprises a simulation spectrum dataset and an in vivo spectrum dataset, the first training phase trains the convolutional neural network using the simulation spectrum dataset, and the second training phase trains the convolutional neural network using the in vivo spectrum dataset.

15. A system for performing frequency-and-phase correction of magnetic resonance spectroscopy (MRS) data to quantify one or more metabolites, the system comprising:
a memory; and
a processor, coupled to the memory, configured to:
receive spectrum data generated using magnetic resonance spectroscopy, wherein the spectrum data relates to a subject's brain and a plurality of metabolite levels;
generate corrected spectrum data by inputting the received spectrum data to a trained convolutional neural network, wherein the trained convolutional neural network simultaneously estimates frequency corrections and phase corrections for the input spectrum data; and
quantify one or more metabolites using the corrected spectrum data.

16. The system of claim 15, wherein the convolutional neural network is trained using a first training phase and a second training phase.

17. The system of claim 16, wherein the first training phase trains the convolutional neural network using a simulation spectrum dataset and the second training phase trains the convolutional neural network using an in vivo spectrum dataset.

18. The system of claim 17, wherein,
the first training phase comprises a) supervised training or b) supervised training and unsupervised training, and
the second training phase comprises unsupervised training.

19. The system of claim 15, wherein the trained convolutional neural network comprises a single trained convolutional neural network, and, during training, the single convolutional neural network is trained to simultaneously estimate frequency corrections and phase corrections for spectrum training data.

20. The system of claim 15, wherein the received spectrum data comprises single voxel MEGA-PRESS MRS data and the quantified one or more metabolites comprise GABA, glutamate, or glutamine.
